(19) United States
(12) Patent Application Publication
(10) Pub. No.: US 2006/0018831 A1
(43) Pub. Date: Jan. 26, 2006

(54) TF binding agent and use thereof

(75) Inventors: Hesse Birger, Gentofte (DK); Andreas Kjaer, Frederiksberg (DK); Henning Ralf Stennicke, Kokkedal (DK); Soren E. Bjorn, Lyngby (DK)

Correspondence Address: NOVO NORDISK, INC.;PATENT DEPARTMENT, 100 COLLEGE ROAD WEST, PRINCETON, NJ, 08540, US

(73) Assignees: Novo Nordisk A/S, Bagsvaerd (DK); H:S RIGSHOSPITALET, Copenhagen (DK)

(21) Appl. No.: 11/186,669

(22) Filed: Jul. 21, 2005

Related U.S. Application Data
(63) Continuation of application No. PCT/DK04/00041, filed on Jan. 22, 2004.
(60) Provisional application No. 60/443,976, filed on Jan. 31, 2003.

(30) Foreign Application Priority Data
Jan. 22, 2003 (DK) PA 2003 00073

(51) Int. Cl. A61K 51/00; C07K 14/755

(52) U.S. Cl. 424/1.69; 530/399; 530/383

(57) ABSTRACT

This invention relates to novel compounds, which bind to tissue factor and the use thereof for diagnostic and/or therapeutic purposes.

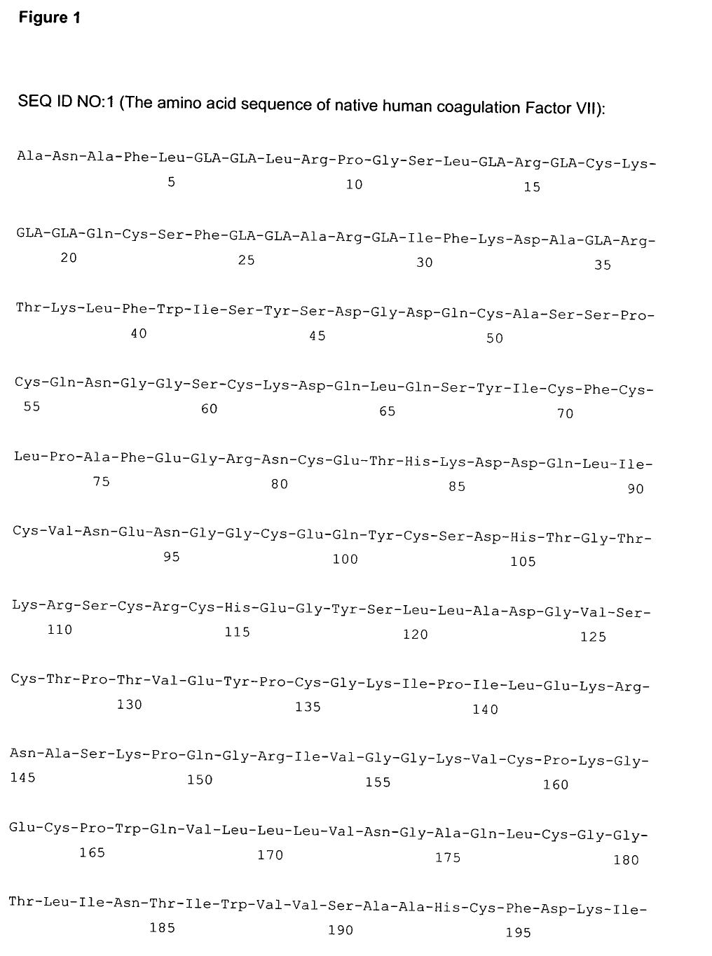

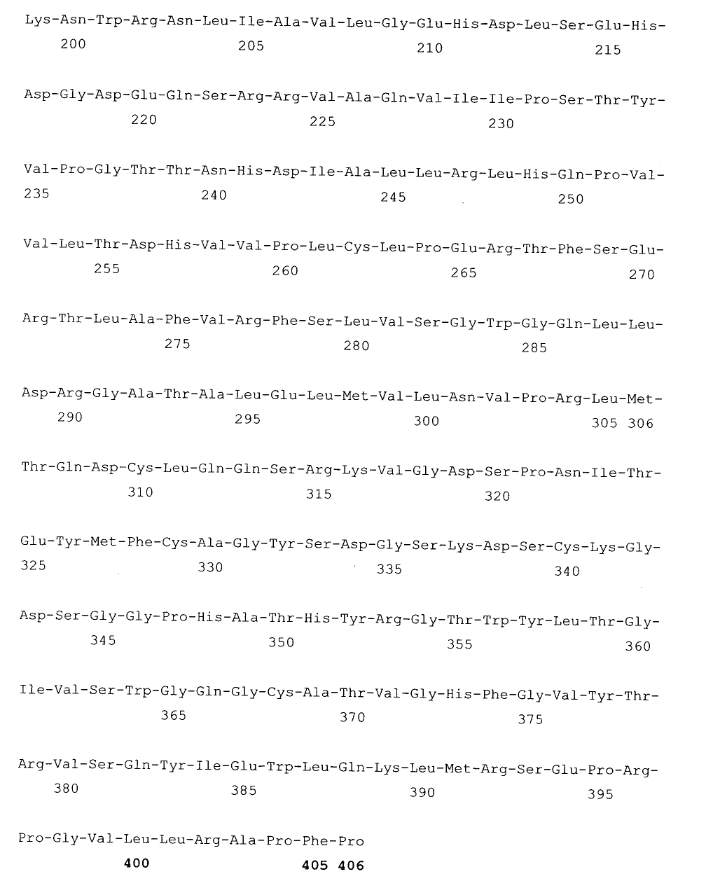

### CROSS-REFERENCE TO RELATED PATENT APPLICATIONS

This patent application is a continuation of International Patent Application PCT/DK2004/000041, filed Jan. 22, 2004, and claims the benefit of priority to Danish Patent Application PA 2003 00073, filed Jan. 22, 2003, and U.S. Provisional Patent Application 60/443,976, filed Jan. 31, 2003.

### FIELD OF THE INVENTION

This invention relates to novel compounds which bind to tissue factor and the use thereof as diagnostic markers. The invention also relates to pharmaceutical compositions comprising the novel compounds as well as their use in the diagnosing, prophylaxis or treatment of diseases or disorders related to pathobiology involving tissue factor (TF) including bleedings, cancer, inflammation, atherosclerosis and ischemia/reperfusion.

### BACKGROUND OF THE INVENTION

Though the medical and surgical management of patients with acute gastro-intestinal bleeding has improved, identification of the bleeding site or organ may sometimes be very difficult, in spite of the current techniques such as endoscopy, angiography, and even explorative laparotomy. The use of ^{99m}Tc-tagged albumin or red blood cells for bleeding scintigraphy may occasionally be helpful in severe, acute gastrointestinal bleeding, but only with bleeding rates exceeding 1 mL/min. Successful localization of the bleeding site with the current scintigraphic tracers is dependent on active bleeding during the first 1-1½ hours after i.v. injection of the tracer, and the recording procedures may be both time consuming (dynamic imaging for 60-90 minutes and subsequent later attempts) and complicated related to the serious condition of many of these patients. The need for an easy, rapid, safe, non-invasive diagnostic tool in acute gastrointestinal bleeding is obvious and might greatly improve the outcome of the patients.

Coagulation is initiated by formation of a complex of FVIIa with its cell surface receptor, tissue factor (TF) at the site of injury. Tissue Factor is a cellular transmembrane receptor for plasma coagulation factor VIIa (FVIIa) and formation of TF/FVIIa complexes on the cell surface triggers the coagulation cascade in vivo. The TF/FVIIa complex efficiently activates coagulation factors IX and X. The resultant protease factor Xa (FXa), activates prothrombin to thrombin, which in turn converts fibrinogen into a fibrin matrix.

Normally, TF is constitutively expressed on the surface of many extravascular cell types that are not in contact with the blood, such as fibroblasts, pericytes, smooth muscle cells and epithelial cells, but not on the surface of cells that come in contact with blood, such as endothelial cells and monocytes. However, TF is also expressed in various pathophysiological conditions where it is believed to be involved in progression of disease states within cancer, inflammation, atherosclerosis and ischemia/reperfusion. Thus, TF is now recognised as a target for therapeutic intervention in conditions associated with increased expression.

FVIIa is a two-chain, 50 kilodalton (kDa) vitamin-K dependent, plasma serine protease which participates in the complex regulation of in vivo haemostasis. FVIIa is generated from proteolysis of a single peptide bond from its single chain zymogen, Factor VII (FVII), which is present at approximately 0.5 μg/ml in plasma. The zymogen is catalytically inactive. The conversion of zymogen FVII into the activated two-chain molecule occurs by cleavage of an internal peptide bond. In the presence of calcium ions, FVIIa binds with high affinity to exposed TF, which acts as a cofactor for FVIIa, enhancing the proteolytic activation of its substrates FVII, Factor IX and FX.

In addition to its established role as an initiator of the coagulation process, TF was recently shown to function as a mediator of intracellular activities either by interactions of the cytoplasmic domain of TF with the cytoskeleton or by supporting the FVIIa-protease dependent signaling. Such activities may be responsible, at least partly, for the implicated role of TF in tumor development, metastasis and angiogenesis. Cellular exposure of TF activity is advantageous in a crisis of vascular damage but may be fatal when exposure is sustained as it is in these various diseased states. Thus, it is critical to regulate the expression of TF function in maintaining the health.

Radiolabelled TF agonists and/or TF antagonists may be valuable for diagnostic imaging with a gamma camera, a PET camera or a PET/CT camera, in particular for the evaluation of TF expression of tumor cells, for grading the malignancy of tumor cells known to express TF receptors, for the monitoring of tumors with TF expression during conventional chemotherapy or radiation therapy. Also TF agonists and/or TF antagonists labelled with alpha- or beta-emitting isotopes could be used for therapy, possibly with bi-specific binding to compounds with chemotherapeutic action, which may be related to the presence of TF receptors. In those cases the diagnostic imaging may be important for the evaluation of tumor response expected after therapy with TF receptor binding drugs.

Also other kinds of diseases with increased expression of surface accessible TF receptors may be observed, maybe inflammatory or auto-immune diseases, where both diagnostic and therapeutic application of radiolabelled TF agonists and/or TF antagonists may become relevant.

One example of a TF antagonist, inactivated FVII (FVIIai) is FVIIa modified in such a way that it is catalytically inactive. Thus, FVIIai is not able to catalyze the conversion of FX to FXa, or FIX to FIXa but still able to bind tightly to TF in competition with active endogenous FVIIa and thereby inhibit the TF function.

International patent applications WO 92/15686, WO 94/27631, WO 96/12800, WO 97/47651 relates to FVIIai and the uses thereof. International patent applications WO 90/03390, WO 95/00541, WO 96/18653, and European Patent EP 500800 describes peptides derived from FVIIa having TF/FVIIa antagonist activity. International patent application WO 01/21661 relates to bivalent inhibitor of FVII and FXa.

Hu Z and Garen A (2001) Proc. Natl. Acad. Sci. USA 98; 12180-12185, Hu Z and Garen A (2000) Proc. Natl. Acad. Sci. USA 97; 9221-9225, Hu Z and Garen A (1999) Proc. Natl. Acad. Sci. USA 96; 8161-8166, and International patent application WO 0102439 relates to immunoconjugates which comprises the Fc region of a human IgG1 immunoglobulin and a mutant FVII polypeptide, that binds to TF but do not initiate blood clotting.

Furthermore, International patent application WO 98/03632 describes bivalent agonists having affinity for one or more G-coupled receptors, and Burgess, L. E. et al., Proc. Natl. Acad. Sci. USA 96, 8348-8352 (July 1999) describes “Potent selective non-peptidic inhibitors of human lung tryptase”.

There is still a need in the art for improved compounds, which efficiently inhibits pathophysiological TF function at relatively low doses and which does not produce undesirable side effects. The present invention provides compounds that act specifically on pathophysiological TF function and at the same time are useful as a diagnostic tool. Alternatively the present invention provides improved compounds, which promote clotting and at the same time are useful as a diagnostic tool.

### DESCRIPTION OF THE INVENTION

The present invention relates to conjugates of TF agonists and/or TF antagonists that are also useful as a diagnostic tool e.g. for scintigraphic localization of the origin of an acute or intermittent gastrointestinal bleeding lesion with or without pathophysiologic function of the coagulation system, or as a diagnostic tool for localization of a tumour with high expression of TH-binding receptor, or for characterization of a known tumour re. its expression and accessibility of TF-binding receptors from the extracellular compartment, or for the diagnosis of malignant dissemination to sentinel lymph nodes. The conjugates bind TF with high affinity and specificity. In one embodiment of the invention the TF binding agent is a TF antagonist, which do not initiate blood coagulation. In one embodiment of the present invention the TF antagonist is FVIIa polypeptides chemically inactivated in the active site. In another embodiment of the present invention the TF antagonist is an antibody against TF. In one embodiment the antibody is a monoclonal antibody. In one embodiment the antibody is a human monoclonal antibody. In one embodiment the antibody is an antibody against human TF.

Methods of preparing human antibodies against human TF is described in International patent application with publication number WO 03/029295 and in International patent application with application number PCT DK03/00741 the content of which is hereby incorporated by reference in its entirety.

The conjugates contain a functional group that provides a detectable emission. This may permit identification and localization of TF, e.g. TF expressing cells, e.g. (tumor) tissue rich in TF expressing target cells or an internal bleeding with exposed TF expressing cells.

The terms “TF antagonist” or “TF antagonists”, as used herein is intended to mean any compound that binds directly to TF and inhibits the conversion of FX to FXa in a FXa generation assay (Assay 1). Examples of TF antagonist includes, but at not limited to FVIIai and inhibitory antibodies against TF.

The terms “TF agonist” or “TF agonists”, as used herein is intended to mean any compound that binds directly to TF without inhibiting the conversion of FX to FXa in a FXa generation assay (Assay 1). In one embodiment of the invention, the TF agonist is FVII In one embodiment of the invention, the TF agonist is FVIIa. In one embodiment of the invention, the TF agonist is a Factor VII-related polypeptide. In one embodiment of the invention, the TF agonist is native human FVIIa or a variant thereof. In a further embodiment the TF agonist is a FVIIa equivalent.

As used herein, “Factor VII” or “FVII” encompasses wild-type Factor VII (i.e., a polypeptide having the amino acid sequence disclosed in U.S. Pat. No. 4,784,950), as well as polypeptide variants of Factor VII exhibiting substantially the same or improved biological activity relative to wild-type Factor VII. The term “Factor VII” is intended to encompass Factor VII polypeptides in their uncleaved (zymogen) form, as well as those that have been proteolytically processed to yield their respective bioactive forms, which may be designated Factor VIIa. Typically, Factor VII is cleaved between residues 152 and 153 to yield Factor VIIa.

The terms “Factor VIIa” or “FVIIa” is intended to encompass, without limitation, polyeptides having the amino acid sequence 1-406 of wild-type human Factor VIIa (as disclosed in U.S. Pat. No. 4,784,950), as well as wild-type Factor VIIa derived from other species, such as, e.g., bovine, porcine, canine, murine, and salmon Factor VIIa. It further encompasses natural allelic variations of Factor VIIa that may exist and occur from one individual to another. Also, degree and location of glycosylation or other post-translation modifications may vary depending on the chosen host cells and the nature of the host cellular environment.

The terms “variant” or “variants”, as used herein, is intended to designate human Factor VII having the sequence of SEQ ID NO: 1, wherein one or more amino acids of the parent protein have been substituted by another amino acid and/or wherein one or more amino acids of the parent protein have been deleted and/or wherein one or more amino acids have been inserted in protein and/or wherein one or more amino acids have been added to the parent protein. Such addition can take place either at the N-terminal end or at the C-terminal end of the parent protein or both. In one embodiment of the invention the variant has a total amont of amino acid substitutions and/or additions and/or deletions independently selected from the group consisting of 1, 2, 3, 4, 5, 6, 7, 8, 9, and 10.

As used herein, “Factor VII-related polypeptides” encompasses polypeptides, including polypeptide variants, in which the Factor VIIa biological activity has been substantially modified or reduced relative to the activity of wild-type Factor VIIa. These polypeptides include, without limitation, Factor VII or Factor VIIa into which specific amino acid sequence alterations have been introduced that modify or disrupt the bioactivity of the polypeptide. The biological activity of Factor VIIa in blood clotting derives from its ability to (i) bind to tissue factor (TF) and (ii) catalyze the proteolytic cleavage of Factor IX or Factor X to produce activated Factor IX or X (Factor IXa or Xa, respectively). For purposes of the invention, Factor VIIa biological activity may be quantified by measuring the ability of a preparation to promote blood clotting using Factor VII-deficient plasma and thromboplastin, as described, e.g., in U.S. Pat. No. 5,997,864. In this assay, biological activity is expressed as the reduction in clotting time relative to a control sample and is converted to “Factor VII units” by comparison with a pooled human serum standard containing 1 unit/ml Factor VII activity. Alternatively, Factor VIIa biological activity may be quantified by (i) measuring the ability of Factor VIIa to produce of Factor Xa in a system comprising TF embedded in a lipid membrane and Factor X. (Persson et al., J. Biol. Chem. 272:19919-19924, 1997); (ii) measuring Factor X hydrolysis in an aqueous system; (iii) measuring its physical binding to TF using an instrument based on surface plasmon resonance (Persson, FEBS Letts. 413:359-363, 1997) and (iv) measuring hydrolysis of a synthetic substrate.

Factor VII variants having substantially the same or improved biological activity relative to wild-type Factor VIIa encompass those that exhibit at least about 25%, preferably at least about 50%, more preferably at least about 75% and most preferably at least about 90% of the specific activity of Factor VIIa that has been produced in the same cell type, when tested in one or more of a clotting assay, proteolysis assay, or TF binding assay as described above. Factor VII variants having substantially reduced biological activity relative to wild-type Factor VIIa are those that exhibit less than about 25%, preferably less than about 10%, more preferably less than about 5% and most preferably less than about 1% of the specific activity of wild-type Factor Vila that has been produced in the same cell type when tested in one or more of a clotting assay, proteolysis assay, or TF binding assay as described above. Factor VII variants having a substantially modified biological activity relative to wild-type Factor VII include, without limitation, Factor VII variants that exhibit TF-independent Factor X proteolytic activity and those that bind TF but do not cleave Factor X.

Variants of Factor VII, whether exhibiting substantially the same or better bioactivity than wild-type Factor VII, or, alternatively, exhibiting substantially modified or reduced bioactivity relative to wild-type Factor VII, include, without limitation, polypeptides having an amino acid sequence that differs from the sequence of wild-type Factor VII by insertion, deletion, or substitution of one or more amino acids.

Non-limiting examples of Factor VII variants having substantially the same biological activity as wild-type Factor VII include S52A-FVIIa, S60A-FVIIa (Lino et al., Arch. Biochem. Biophys. 352: 182-192, 1998); FVIIa variants exhibiting increased proteolytic stability as disclosed in U.S. Pat. No. 5,580,560; Factor VIIa that has been proteolytically cleaved between residues 290 and 291 or between residues 315 and 316 (Mollerup et al., Biotechnol. Bioeng. 48:501-505, 1995); oxidized forms of Factor VIIa (Kornfelt et al., Arch. Biochem. Biophys. 363:43-54, 1999); Other non-limiting examples of Factor VII variants include FVII variants as disclosed in International patent application WO 02/077218; and FVII variants as disclosed in WO 02/38162 (Scripps Research Institute); FVII variants having a modified Gla-domain and exhibiting an enhanced membrane binding as disclosed in WO 99/20767 and WO 00/66753 (University of Minnesota); and FVII variants as disclosed in WO 01/58935 (Maxygen ApS) and WO 02/02764 (University of Minnesota).

Non-limiting examples of Factor VII variants having substantially reduced or modified biological activity relative to wild-type Factor VII include R152E-FVIIa (Wildgoose et al., Biochem 29:3413-3420, 1990), S344A-FVIIa (Kazama et al., J. Biol. Chem. 270:66-72, 1995), FFR-FVIIa (Hoist et al., Eur. J. Vasc. Endovasc. Surg. 15:515-520, 1998), and Factor VIIa lacking the Gla domain, (Nicolaisen et al., FEBS Letts. 317:245-249, 1993).

Non-limiting examples of FVII variants having increased biological activity compared to wild-type FVIIa include FVII variants as disclosed in International patent applications WO 01/83725, WO 02/22776, WO 03/027147, WO 03/037932, WO 04/000366, WO 02/38162 (Scripps Research Institute), International patent application with application number PCT/DK03/00625; and FVIIa variants with enhanced activity as disclosed in JP 2001061479 (Chemo-Sero-Therapeutic Res Inst.).

Examples of factor VII or factor VII-related polypeptides include, without limitation, wild-type Factor VII, L305V-FVII, L305V/M306D/D309S-FVII, L3051-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII, L305V/K337A-FVII, L305V/V158D-FVII, L305V/E296V-FVII, L305V/M298Q-FVII, L305V/V158T-FVII, L305V/K337A/M158T-FVII, L305V/K337A/M298Q-FVII, L305V/K337A/E296V-FVII, L305V/K337A/V158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V158T/E296V/M298Q/K337A-FVII, S314E/K316H-FVII, S314E/K316Q-FVII, S314E/L305V-FVII, S314E/K337A-FVII, S314E/L158D-FVII, S314E/E296V-FVII, S314E/M298Q-FVII, S314E/L158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/E158T-FVII, S314E/L305V/K337A/M298Q-FVII, S314E/L305V/K337A/E296V-FVII, S314E/L305V/K337A/E158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A-FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/M158T-FVII, K316Q/L305V/K337A/M298Q-FVII, K316Q/L305V/K337A/E296V-FVII, K316Q/L305V/K337A/M158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A-FVII, K316Q/L305V/V158D/E296V/M298Q/K337A-FVII, K316Q/L305V/V158T/E296V/M298Q/K337A-FVII, S52A-Factor VII, S60A-Factor VII; R152E-Factor VII, S344A-Factor VII, Factor VIIa lacking the GIa domain; and P11Q/K33E-FVII, T106N-FVII, K143N/N145T-FVII, V253N-FVII, R290N/A292T-FVII, G291N-FVII, R315NN317T-FVII, K143N/N145T/R315N/V317T-FVII; and FVII having substitutions, additions or deletions in the amino acid sequence from 233Thr to 240Asn, FVII having substitutions, additions or deletions in the amino acid sequence from 304Arg to 329Cys.

Currently no TF antagonists have been developed and marketed for therapeutic use in humans. Known therapeutic strategies include monoclonal antibodies, catalytically impaired FVIIa mutants and chemical inactivated FVIIa. Native FVIIa binds TF with high affinity and most mutants with amino acid substitutions and monoclonal antibodies are expected to bind with a similar or decreased affinity. A low affinity for TF may limit the effective use in the clinic. Chemically inactivated FVIIa has been reported to possess a modestly increased affinity for TF as compared to native FVIIa.

The reported inactive mutants of FVIIa as well as the chemically inactivated FVIIa is expected to have short half lives comparable to that of circulating native FVII, i.e. 2-3 hours, which may limit the effective use in the clinic.

The present invention relates to radiolabelled TF agonists and/or TF antagonists conjugated to a compound containing a radionuclide. It is to be understood, that the conjugate binds to and kills or arrest the growth of the TF presenting cells. The terms “TF presenting cell” or “TF presenting cells” as used herein refers to the presence of TF protein on a cell surface plasma membrane. TF may be located in the membranes of cells where it was synthesized by protein synthesis or it may have accumulated after it has been synthesized and shed by other cells.

The inactivation of the FVIIa proteolytic activity is obtained in vitro by covalent active site inhibitors e.g. chloromethyl ketones. The conjugate has very high affinity for TF due to the increased affinity of the chemically inactivated FVIIa moiety as compared to the binding of native FVIIa. The high affinity will provide a more efficacious and safe treatment of a patient in need thereof. The conjugate may also have an even higher affinity for TF due an avidity effect introduced by the presence of FVIIai dimers, trimers or multimers with multible TF binding sites.

In one embodiment the present invention relates to chemically inactivated FVII molecules in which the active site is covalently modified by:
1) A covalent active site inhibitor for delivery of radionuclides. In this scenario radionuclides may selectively accumulate at the surface or inside the targeted cell.
2) A covalent active site inhibitor comprising a radionuclide. In this scenario radionuclides could be used in a diagnostic imaging of the targeted cell.

While full length FVII does represent the preferred embodiment for drug delivery in 1, 2 and 3, this does not exclude the use of FVII (des-Gla) or any other TF-binding FVII derived protein including truncated forms, analogs, derivatives and fusion proteins (monomers, homo- or heterodimers or multimers). The different affinity of such molecules for TF may provide a method for reducing the potentially undesirable effect of a diagnostic compound on general haemostasis.

In a first aspect, the present invention relates to a compound having the formula A-(LM)-C, wherein A is a TF antagonist or TF agonist; LM is an optional linker moiety; C is a compound comprising a radionuclide. In one embodiment LM is present. In one embodiment LM is absent.

In a second aspect, the present invention relates to a pharmaceutical composition comprising an amount of the compound having the formula A-(LM)-C, wherein A is a TF antagonist or TF agonist; LM is an optional linker moiety; C is a compound comprising a radionuclide; and a pharmaceutically acceptable carrier or diluent.

In a third aspect, the present invention relates to a compound for use as a medicament having the formula A-(LM)-C, wherein A is a TF antagonist or TF agonist; LM is an optional linker moiety; C is a compound comprising a radionuclide; and wherein the compound binds to TF and inhibits TF function.

In a further aspect, the present invention relates to the use of a compound having the formula A-(LM)-C, wherein A is a TF antagonist or TF agonist; LM is an optional linker moiety; C is a compound comprising a radionuclide; for the manufacture of a medicament for diagnosing, preventing or treating disease or disorder associated with pathophysiological TF function. In a preferred embodiment the use is diagnostic application of administration to patients of a compound having the formula A-(LM)-C, wherein A is a TF antagonist or TF agonist; LM is an optional linker moiety; C is a compound comprising a radionuclide.

In a further aspect, the present invention relates to the use of a compound having the formula A-(LM)-C, wherein A is a TF antagonist or TF agonist; LM is an optional linker moiety; C is a compound comprising a radionuclide; for the manufacture of a medicament for diagnostic imaging of a disease or disorder associated with pathophysiological TF function. In one embodiment the medicament is used for the diagnostic imaging of a disease or disorder associated with pathophysiological TF function in internal organs of a mammal, such as internal bleedings or cancers.

The term “disease or disorder associated with pathophysiological TF function” as used herein means any disease or disorder, where TF is involved. This includes, but are not limited to diseases or disorders related to TF-mediated coagulation activity, e.g. bleeding disorder such as clotting factor deficiencies (e.g. haemophilia A and B or deficiency of coagulation Factors XI or VII) or clotting factor inhibitors, excessive bleeding occurring in subjects with a normally functioning blood clotting cascade (no clotting factor deficiencies or inhibitors against any of the coagulation factors), e.g. caused by a defective platelet function, thrombocytopenia or von Willebrand's disease, bleedings in connection with surgery and other forms of tissue damage incl. trauma, bleedings in internal organs, thrombotic or coagulopathic related diseases or disorders or diseases or disorders such as inflammatory responses and chronic thromboembolic diseases or disorders associated with fibrin formation, including vascular disorders such as deep venous thrombosis, arterial thrombosis, post surgical thrombosis, coronary artery bypass graft (CABG), percutaneous transdermal coronary angioplastry (PTCA), stroke, cancer, tumor growth, tumor metastasis, angiogenesis, thrombolysis, arteriosclerosis and restenosis following angioplastry, acute and chronic indications such as inflammation, septic chock, septicemia, hypotension, adult respiratory distress syndrome (ARDS), disseminated intravascular coagulopathy (DIC), pulmonary embolism, platelet deposition, myocardial infarction, or the prophylactic treatment of mammals with atherosclerotic vessels at risk for thrombosis, and other diseases. The disease or disorder associated with pathophysiological TF function are not limited to in vivo coagulopatic disorders such as those named above, but includes ex vivo TF/FVIIa related processes such as coagulation that may result from the extracorporeal circulation of blood, including blood removed in-line from a patient in such processes as dialysis procedures, blood filtration, or blood bypass during surgery.

In one embodiment the disease or disorder associated with pathophysiological TF function is one, where TF is exposed to the blood. In one embodiment the disease or disorder associated with pathophysiological TF function is one, where TF is expressed to a level higher than under normal physiological conditions.

“Treatment” means the administration of an effective amount of a therapeutically active compound of the invention with the purpose of preventing any symptoms or disease state to develop or with the purpose of curing or easing such symptoms or disease states already developed. The term “treatment” is thus meant to include prophylactic treatment.

The terms “cancer or “tumor” are to be understood as referring to all forms of neoplastic cell growth, including both cystic and solid tumors, bone and soft tissue tumors, including both benign and malignant tumors, including tumors in anal tissue, bile duct, bladder, blood cells, bone, bone (secondary), bowel (colon & rectum), brain, brain (secondary), breast, breast (secondary), carcinoid, cervix, children's cancers, eye, gullet (oesophagus), head & neck, kaposi's sarcoma, kidney, larynx, leukaemia (acute lymphoblastic), leukaemia (acute myeloid), leukaemia (chronic lymphocytic), leukaemia (chronic myeloid), leukaemia (other), liver, liver (secondary), lung, lung (secondary), lymph nodes (secondary), lymphoma (hodgkin's), lymphoma (non-hodgkin's), melanoma, mesothelioma, myeloma, ovary, pancreas, penis, prostate, skin, soft tissue sarcomas, stomach, testes, thyroid, unknown primary tumor, vagina, vulva, womb (uterus).

Soft tissue tumors include Benign schwannoma Monosomy, Desmoid tumor, Lipoblastoma, Lipoma, Uterine leiomyoma, Clear cell sarcoma, Dermatofibrosarcoma, Ewing sarcoma, Extraskeletal myxoid chondrosarcoma, Liposarcoma myxoid, Liposarcoma, well differentiated, Alveolar rhabdomyosarcoma, and Synovial sarcoma.

Specific bone tumor include Nonossifying Fibroma, Unicameral bone cyst, Enchondroma, Aneurysmal bone cyst, Osteoblastoma, Chondroblastoma, Chondromyxofibroma, Ossifying fibroma and Adamantinoma, Giant cell tumor, Fibrous dysplasia, Ewing's Sarcoma, Eosinophilic Granuloma, Osteosarcoma, Chondroma, Chondrosarcoma, Malignant Fibrous Histiocytoma, and Metastatic Carcinoma.

Leukaemias referes to cancers of the white blood cells which are produced by the bone marrow. This includes but are not limited to the four main types of leukaemia; acute lymphoblastic (ALL), acute myeloblastic (AML), chronic lymphocytic (CLL) and chronic myeloid (CML).

As used herein the term “bleeding disorder” reflects any defect, congenital, acquired or induced, of cellular or molecular origin that is manifested in bleedings. Examples are clotting factor deficiencies (e.g. haemophilia A and B or deficiency of coagulation Factors XI or VII), clotting factor inhibitors, defective platelet function, thrombocytopenia or von Willebrand's disease.

The term “bleeding episodes” is meant to include uncontrolled and excessive bleeding which is a major problem both in connection with surgery and other forms of tissue damage. Uncontrolled and excessive bleeding may occur in subjects having a normal coagulation system and subjects having coagulation or bleeding disorders. Clotting factor deficiencies (haemophilia A and B, deficiency of coagulation factors XI or VII) or clotting factor inhibitors may be the cause of bleeding disorders. Excessive bleedings also occur in subjects with a normally functioning blood clotting cascade (no clotting factor deficiencies or -inhibitors against any of the coagulation factors) and may be caused by a defective platelet function, thrombocytopenia or von Willebrand's disease. In such cases, the bleedings may be likened to those bleedings caused by haemophilia because the haemostatic system, as in haemophilia, lacks or has abnormal essential clotting “compounds” (such as platelets or von Willebrand factor protein) that causes major bleedings. In subjects who experience extensive tissue damage in association with surgery or vast trauma, the normal haemostatic mechanism may be overwhelmed by the demand of immediate haemostasis and they may develop bleeding in spite of a normal haemostatic mechanism. Achieving satisfactory haemostasis also is a problem when bleedings occur in organs such as the brain, inner ear region and eyes with limited possibility for surgical haemostasis. The same problem may arise in the process of taking biopsies from various organs (liver, lung, tumour tissue, gastrointestinal tract) as well as in laparoscopic surgery. Common for all these situations is the difficulty to provide haemostasis by surgical techniques (sutures, clips, etc.) which also is the case when bleeding is diffuse (haemorrhagic gastritis and profuse uterine bleeding). Acute and profuse bleedings may also occur in subjects on anticoagulant therapy in whom a defective haemostasis has been induced by the therapy given. Such subjects may need surgical interventions in case the anticoagulant effect has to be counteracted rapidly. Radical retropubic prostatectomy is a commonly performed procedure for subjects with localized prostate cancer. The operation is frequently complicated by sigrnificant and sometimes massive blood loss. The considerable blood loss during prostatectomy is mainly related to the complicated anatomical situation, with various densely vascularized sites that are not easily accessible for surgical haemostasis, and which may result in diffuse bleeding from a large area. Another situation that may cause problems in the case of unsatisfactory haemostasis is when subjects with a normal haemostatic mechanism are given anticoagulant therapy to prevent thromboembolic disease. Such therapy may include heparin, other forms of proteoglycans, warfarin or other forms of vitamin K-antagonists as well as aspirin and other platelet aggregation inhibitors.

In one embodiment of the invention, the bleeding is associated with haemophilia. In another embodiment, the bleeding is associated with haemophilia with aquired inhibitors. In another embodiment, the bleeding is associated with thrombocytopenia. In another embodiment, the bleeding is associated with von Willebrand's disease. In another embodiment, the bleeding is associated with severe tissue damage. In another embodiment, the bleeding is associated with severe trauma. In another embodiment, the bleeding is associated with surgery. In another embodiment, the bleeding is associated with laparoscopic surgery. In another embodiment, the bleeding is associated with haemorrhagic gastritis. In another embodiment, the bleeding is profuse uterine bleeding. In another embodiment, the bleeding is occurring in organs with a limited possibility for mechanical haemostasis. In another embodiment, the bleeding is occurring in the brain, inner ear region or eyes. In another embodiment, the bleeding is associated with the process of taking biopsies. In another embodiment, the bleeding is associated with anticoagulant therapy.

In a further aspect, the present invention relates to a method for diagnosing, preventing or treating disease or disorder associated with pathophysiological TF function, said method comprising contacting a TF presenting cell with a compound having the formula A-(LM)-C, wherein A is a TF antagonist or TF agonist; LM is an optional linker moiety; C is a compound comprising a radionuclide.

In one embodiment of the invention A is a TF antagonist. In one embodiment of the invention A is a TF agonist. In one embodiment of the invention the TF agonist is native human FVIIa or a variant thereof.

In one embodiment of the invention the disease or disorder associated with pathophysiological TF function are bleedings, deep venous thrombosis, arterial thrombosis, post surgical thrombosis, coronary artery bypass graft (CABG), percutaneous transdermal coronary angioplastry (PTCA), stroke, cancer, tumor growth, tumour metastasis, angiogenesis, ischemia/reperfusion, rheumatoid arthritis, thrombolysis, arteriosclerosis and restenosis following angioplastry, acute and chronic indications such as inflammation, septic chock, septicemia, hypotension, adult respiratory distress syndrome (ARDS), disseminated intravascular coagulopathy (DIC), pulmonary embolism, platelet deposition, myocardial infarction, or the prophylactic treatment of mammals with atherosclerotic vessels at risk for thrombosis

In one embodiment of the invention, A in the compound having the formula A-(LM)-C is an inactive FVIIa polypeptide.

In a further embodiment of the invention, A in the compound having the formula A-(LM)-C is native human FVIIa or a fragment thereof catalytically inactivated in the active site.

In a further embodiment of the invention, A in the compound having the formula A-(LM)-C is native human FVIIa catalytically inactivated in the active site.

In a further embodiment of the invention, C or (LM)-C in the compound having the formula A-(LM)-C is conjugated to the active site of the FVIIa polypeptide.

In a further embodiment of the invention, A in the compound having the formula A-(LM)-C is an inactive FVIIa polypeptide catalytically inactivated in the active site with a chloromethyl ketone inhibitor independently selected from the group consisting of Phe-Phe-Arg chloromethyl ketone, Phe-Phe-Arg chloromethylketone, D-Phe-Phe-Arg chloromethyl ketone, D-Phe-Phe-Arg chloromethylketone Phe-Pro-Arg chloromethylketone, D-Phe-Pro-Arg chloromethylketone, Phe-Pro-Arg chloromethylketone, D-Phe-Pro-Arg chloromethylketone, L-Glu-Gly-Arg chloromethylketone and D-Glu-Gly-Arg chloromethylketone, Dansyl-Phe-Phe-Arg chloromethyl ketone, Dansyl-Phe-Phe-Arg chloromethylketone, Dansyl-D-Phe-Phe-Arg chloromethyl ketone, Dansyl-D-Phe-Phe-Arg chloromethylketone, Dansyl-Phe-Pro-Arg chloromethylketone, Dansyl-D-Phe-Pro-Arg chloromethylketone, Dansyl-Phe-Pro-Arg chloromethylketone, Dansyl-D-Phe-Pro-Arg chloromethylketone, Dansyl-L-Glu-Gly-Arg chloromethylketone and Dansyl-D-GluGly-Arg chloromethylketone.

In a further embodiment of the invention, LM in the compound having the formula A-(LM)-C comprises a chloromethyl ketone inhibitor independently selected from the group consisting of Phe-Phe-Arg chloromethyl ketone, Phe-Phe-Arg chloromethylketone, D-Phe-Phe-Arg chloromethyl ketone, D-Phe-Phe-Arg chloromethylketone Phe-Pro-Arg chloromethylketone, D-Phe-Pro-Arg chloromethylketone, Phe-Pro-Arg chloromethylketone, D-Phe-Pro-Arg chloromethylketone, L-Glu-Gly-Arg chloromethylketone and D-Glu-Gly-Arg chloromethylketone, Dansyl-Phe-Phe-Arg chloromethyl ketone, Dansyl-Phe-Phe-Arg chloromethylketone, Dansyl-D-Phe-Phe-Arg chloromethyl ketone, Dansyl-D-Phe-Phe-Arg chloromethylketone, Dansyl-Phe-Pro-Arg chloromethylketone, Dansyl-D-Phe-Pro-Arg chloromethylketone, Dansyl-Phe-Pro-Arg chloromethylketone, Dansyl-D-Phe-Pro-Arg chloromethylketone, Dansyl-L-Glu-Gly-Arg chloromethylketone and Dansyl-D-Glu-Gly-Arg chloromethylketone, wherein the inactive FVIIa polypeptide is catalytically inactivated in the active site with said chloromethyl ketone inhibitor.

In a further embodiment of the invention, A in the compound having the formula A-(LM)-C is an antibody against TF.

In a further embodiment of the invention, A in the compound having the formula A-(LM)-C is a human monoclonal antibody against human TF.

In a further embodiment of the invention, C in the compound having the formula A-(LM)-C is a compound containing radionuclides. In another specific embodiment C in the compound having the formula A-(LM)-C comprise I¹²⁵.

In one embodiment C in the compound having the formula A-(LM)-C is a compound containing a radionuclide that is a gamma emitter.

In one embodiment C in the compound having the formula A-(LM)-C is a compound containing a radionuclide that is a beta emitter.

In one embodiment C in the compound having the formula A-(LM)-C is a compound containing a radionuclide that is an alpha emitter.

In one embodiment C in the compound having the formula A-(LM)-C is a compound containing the radionuclide Tc-99m.

In one embodiment C in the compound having the formula A-(LM)-C is a compound containing the radionuclide 188-Re.

In one embodiment C in the compound having the formula A-(LM)-C is a compound containing the radionuclide 123-I.

In one embodiment C in the compound having the formula A-(LM)-C is a compound containing the radionuclide 131-I.

In one embodiment C in the compound having the formula A-(LM)-C is a compound containing the radionuclide Indium-111.

In one embodiment C in the compound having the formula A-(LM)-C is a compound containing the radionuclide Fluorine-18.

In a further embodiment of the invention, C in the compound having the formula A-(LM)-C comprises a protein or peptide.

In a further embodiment of the invention, C or (LM)-C in the compound having the formula A-(LM)-C is conjugated at the glycosylation side chains of A.

In a further embodiment of the invention, C or (LM)-C in the compound having the formula A-(LM)-C is conjugated to a free sulfhydryl group present on A.

In a further embodiment the compound having the formula A-(LM)-C comprises more than one binding site for TF. In one embodiment the compound is a dimer. In one embodiment the compound is a trimer. In one embodiment the compound is a tetramer. In one embodiment the compound is a pentamer. In one embodiment the compound is a hexamer.

In a further embodiment of the invention, LM in the compound having the formula A-(LM)-C comprises an amino acid sequence.

In a further embodiment of the invention, LM in the compound having the formula A-(LM)-C comprises an amino acid sequence of Gly-Gly.

In a further embodiment of the invention, LM in the compound having the formula A-(LM)-C comprises a molecule selected from the group consisting of straight or branched C₁₋₅₀-alkyl, straight or branched C₂₋₅₀-alkenyl, straight or branched C₂₋₅₀-alkynyl, a 1 to 50-membered straight or branched chain comprising carbon and at least one N, O or S atom in the chain, C₃₋₈cycloalkyl, a 3 to 8-membered cyclic ring comprising carbon and at least one N, O or S atom in the ring, aryl, heteroaryl, amino acid, the structures optionally substituted with one or more of the following groups: H, hydroxy, phenyl, phenoxy, benzyl, thienyl, oxo, amino, C₁₋₄-alkyl, —CONH₂, —CSNH₂, C₁₋₄ monoalkylamino, C₁₋₄ dialkylamino, acylamino, sulfonyl, carboxy, carboxamido, halogeno, C₁₋₆ alkoxy, C₁₋₆ alkylthio, trifluoroalkoxy, alkoxycarbonyl, haloalkyl.

In a further embodiment of the invention, LM in the compound having the formula A-(LM)-C comprises a chemical bond, which is breakable by chemical reduction.

In a further embodiment of the invention, LM in the compound having the formula A-(LM)-C comprises a disulpide bond. In one embodiment the disulphide bond is between two cysteines.

In a further embodiment of the invention, LM in the compound having the formula A-(LM)-C comprises a cleavage site for enzyme hydrolysis. In one embodiment the enzyme is a lipase. In another embodiment the enzyme is a protease.

In a further embodiment of the invention, LM in the compound having the formula A-(LM)-C comprises a cleavage site for protease hydrolysis, wherein the protease is selected from the group consisting of cathepsin B, cathepsin D, cathepsin E, cathepsin G, cathepsin H, cathepsin L, cathepsin N, cathepsin S, cathepsin T, cathepsin K, and legumain. In a specific embodiment, the protease is cathepsin B.

In a further embodiment of the invention, LM in the compound having the formula A-(LM)-C comprises the amino acid sequence Phe-Arg.

The terms “compound comprising a radionuclide” as used herein refers to any compound comprising a radionuclide, e.g. compounds comprising radionuclides for the primary purpose of diagnostic imaging of the target cells. The term is intended to include radioactive isotopes or radionuclides (e.g. I131, I125, I123, In111, Y90, Tc99m, Re186, PET tracers such as 11-C, 13-N, 15-O, and 18-F and other radioisotopes suited for imaging with conventional gamma camera, non-imaging probes, positron emission tomographic cameras, and other in vivo and in vitro imaging cameras or radioactivity-recording devices).

Imaging may be performed as a conventional planar gamma camera study, a tomographic gamma camera study (SPECT), a gamma camera study combined with low-dose CT scanning or a positron emission tomography (PET) scan with or without combination with CT-scanning. The type of radionuclide labelled to the TF agonist or antagonist will depend on the purpose of the study and the equipment used; In case of acute gastro-intestinal bleeding, it will be important to label the TF agonist or antagonist with ^{99m}Tc, which is always available in a department of nuclear medicine. If the drug is intended for longer lasting acquisitions such as imaging of low grade bleeding conditions, ¹¹¹indium may be better suited, and for cancer imaging on a gamma camera ¹¹¹indium or ¹³¹iodine with an even longer half life may be necessary. ¹³¹ Iodine is at the same time a candidate for therapeutic application with its combination of beta- and gamma-emitting isotopes. If used with PET scanning ¹⁸F-fluorine labelling may be the preferable isotope, if a shorter half life is sufficient. Other isotopes may be relevant if longer time is needed for binding to tumor cells.

Diagnostic compounds may include, but are not limited to radionuclides. Radionuclides may include, but are not limited to radiometals such as yttrium which emits a high energy beta particle, and I¹²⁵ that emits Auger electrons, that may be absorbed by adjacent TF presenting cells. The methods for coupling ligands or targeting molecules with therapeutic compounds are well known to those skilled in the art (See, for example, conjugates as reviewed by Ghetie et al., 1994, Pharmacol. Ther. 63:209-34; U.S. Pat. No. 5,789,554, the disclosure of which is herein incorporated by reference). Often such methods utilize one of several available heterobifunctional reagents used for coupling or linking molecules.

Radionuclides useful within the present invention include gamma-emitters, positron-emitters, Auger electron-emitters, X-ray emitters and fluorescence-emitters, with beta- or alphaemitters preferred for therapeutic use. Radionuclides are well-known in the art and include 123-I, 125-I, 130-I, 131-I, 133-I, 135-I 47-Sc, 72-As, 72-Se, 90-Y, 88-Y, 97-Ru, 100-Pd, 101m-Rh, 119-Sb, 128-Ba, 197-Hg, 211-At, 212-Bi, 153-Sm, 169-Eu, 212-Pb, 109-Pd, 111-In, 67-Ga, 68-Ga, 64-Cu, 67-Cu, 75-Br, 76-Br, 77-Br, 99m-Tc, 11-C, 13-N, 15-O, 166-Ho and 18-F. Preferred therapeutic radionuclides include 188-Re, 186-Re, 203-Pb, 212-Pb, 212-Bi, 109-Pd, 64-Cu, 67-Cu, 90-Y, 99m-Tc, 123-I, 125-I, 131-I, 77-Br, 211-At, 97-Ru, 105-Rh, 198-Au and 199-Ag, 166-Ho or 177-Lu.

In one embodiment C in the compound having the formula A-(LM)-C is a compound containing the radionuclide selected from the group consisting of I-131, I-125, I-123, In-111, Y-90, Tc-99m, Re-186, 11-C, 13-N, 15-O, and 18-F.

In one embodiment C in the compound having the formula A-(LM)-C is a compound containing the radionuclide selected from the group consisting of 123-I, 125-I, 130-I, 131-I, 133-I, 135-I 47-Sc, 72-As, 72-Se, 90-Y, 88-Y, 97-Ru, 100-Pd, 101 m-Rh, 119-Sb, 128-Ba, 197-Hg, 211-At, 212-Bi, 153-Sm, 169-Eu, 212-Pb, 109-Pd, 111-In, 67-Ga, 68-Ga, 64-Cu, 67-Cu, 75-Br, 76-Br, 77-Br, 99m-Tc, 11-C, 13-N, 15-O, 166-Ho and 18-F.

In one embodiment C in the compound having the formula A-(LM)-C is a compound containing the radionuclide selected from the group consisting of 188-Re, 186-Re, 203-Pb, 212-Pb, 212-Bi, 109-Pd, 64-Cu, 67-Cu, 90-Y, 99m-Tc, 123-I, 125-I, 131-I, 77-Br, 211-At, 97-Ru, 105-Rh, 198-Au and 199-Ag, 166-Ho or 177-Lu.

The terms “Nucleic acid sequence” or “nucleotide sequence” as used herein refers to an oligonucleotide, nucleotide, or polynucleotide, and fragments or portions thereof, and to DNA or RNA of genomic or synthetic origin which may be single- or double-stranded, and represent the sense or antisense strand. Similarly, “amino acid sequence” as used herein refers to an oligopeptide, peptide, polypeptide, or protein sequence, and fragments or portions thereof, and to naturally occurring or synthetic molecules.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

The terms “FVIIa polypeptide” or “FVIIa polypeptides” as used herein means native Factor VIIa, as well as equivalents of Factor VIIa that contain one or more amino acid sequence alterations relative to native Factor VIIa (i.e., Factor VII variants), and/or contain truncated amino acid sequences relative to native Factor VIIa (i.e., Factor VIIa fragments). Such equivalents may exhibit different properties relative to native Factor VIIa, including stability, phospholipid binding, altered specific proteolytic activity, and the like.

As used herein, “Factor VII equivalent” encompasses, without limitation, equivalents of Factor VIIa exhibiting TF binding activity. The term “TF binding activity” as used herein means the ability of a FVIIa polypeptide or TF antagonist to inhibit the binding of recombinant human ¹²⁵I-FVIIa to cell surface human TF. The TF binding activity may be measured as described in Assay 3.

Factor VII equivalents also includes proteolytically inactive variants of FVIIa. In one embodiment of the invention the FVIIa polypeptide is human FVIIa, which has an amino acid substitution of the lysine corresponding to position 341 of SEQ ID NO: 1.

In one embodiment of the invention the FVIIa polypeptide is human FVIIa, which has an amino acid substitution of the serine corresponding to position 344 of SEQ ID NO: 1.

In one embodiment of the invention the FVIIa polypeptide is human FVIIa, which has an amino acid substitution of the aspartic acid corresponding to position 242 of SEQ ID NO: 1.

In one embodiment of the invention the FVIIa polypeptide is human FVIIa, which has an amino acid substitution of the histidine corresponding to position 193 of SEQ ID NO: 1.

In one embodiment the FVIIa polypeptide is FVII-(K341A)

In one embodiment the FVIIa polypeptide is FVII-(S344A)

In one embodiment the FVIIa polypeptide is FVII-(D242A)

In one embodiment the FVIIa polypeptide is FVII-(H193A)

The terminology for specific amino acid substitutions used herein are as follows. The first letter represent the amino acid naturally present at a position of SEQ ID NO: 1. The following number represent the position in SEQ ID NO: 1. The second letter represent the different amino acid substituting for the natural amino acid. An example is FVII-(K341A), where a lysine at position 341 of SEQ ID NO: 1 is replaced by an alanine. In another example, FVII-(K341A/S344A), the lysine at position 341 of SEQ ID NO: 1 is replaced by an alanine and the serine in position 344 of SEQ ID NO: 1 is replaced by an alanine in the same Factor VII polypeptide.

The term “active site” and the like when used herein with reference to FVIIa refer to the catalytic and zymogen substrate binding site, including the “S₁” site of FVIIa as that term is defined by Schecter, I. and Berger, A., (1967) Biochem. Biophys. Res. Commun. 7:157-162.

The term “TF-mediated coagulation activity” means coagulation initiated by TF through the formation of the TF/FVIIa complex and its activation of FIX and Factor X to FIXa and FXa, respectively. TF-mediated coagulation activity is measured in a FXa generation assay. The term “FXa generation assay” as used herein is intended to mean any assay where activation of FX is measured in a sample comprising TF, FVIIa, FX, calcium and phospholipids. An example of a FXa generation assay is described in assay 1.

A TF/FVIIa mediated or associated process or event, or a process or event associated with TF-mediated coagulation activity, is any event, which requires the presence of TF/FVIIa.

Such processes or events include, but are not limited to, formation of fibrin which leads to thrombus formation; platelet deposition; proliferation of smooth muscle cells (SMCs) in the vessel wall, such as, for example, in intimal hyperplasia or restenosis, which is thought to result from a complex interaction of biological processes including platelet deposition and thrombus formation, release of chemotactic and mitogenic factors, and the migration and proliferation of vascular smooth muscle cells into the intima of an arterial segment; and deleterious events associated with post-ischemic reperfusion, such as, for example, in patients with acute myocardial infarction undergoing coronary thrombolysis.

The no-reflow phenomenon, that is, lack of uniform perfusion to the microvasculature of a previously ischemic tissue has been described for the first time by Krug et al., (Circ. Res. 1966; 19:57-62).

The general mechanism of blood clot formation is reviewed by Ganong, in Review of Medical Physiology, 13^{th} ed., Lange, Los Altos Calif., pp 411-414 (1987). Coagulation requires the confluence of two processes, the production of thrombin which induces platelet aggregation and the formation of fribrin which renders the platelet plug stable. The process comprises several stages each requiring the presence of discrete proenzymes and profactors. The process ends in fibrin crosslinking and thrombus formation. Fibrinogen is converted to fibrin by the action of thrombin. Thrombin, in turn, is formed by the proteolytic cleavage of prothrombin. This proteolysis is effected by FXa which binds to the surface of activated platelets and in the presence of FVa and calcium, cleaves prothrombin. TF/FVIIa is required for the proteolytic activation of FX by the extrinsic pathway of coagulation. Therefore, a process mediated by or associated with TF/FVIIa, or an TF-mediated coagulation activity includes any step in the coagulation cascade from the formation of the TF/FVIIa complex to the formation of a fibrin platelet clot and which initially requires the presence of TF/FVIIa. For example, the TF/FVIIa complex initiates the extrinsic pathway by activation of FX to FXa, FIX to FIXa, and additional FVII to FVIIa. TF/FVIIa mediated or associated process, or TF-mediated coagulation activity can be conveniently measured employing standard assays such as those described in Roy, S., (1991) J. Biol. Chem. 266:4665-4668, and O'Brien, D. et al., (1988) J. Clin. Invest. 82:206-212 for the conversion of FX to FXa in the presence of TF/FVIIa and other necessary reagents.

It should be noted that peptides, proteins and amino acids as used herein can comprise or refer to “natural”, i.e., naturally occurring amino acids as well as “non-classical” D-amino acids including, but not limited to, the D-isomers of the common amino acids, α-isobutyric acid, 4-aminobutyric acid, hydroxyproline, sarcosine, citrulline, cysteic acid, t-butylglycine, tbutylalanine, phenylglycine, cyclohexylalanine, β-alanine, designer amino acids such as β-methyl amino acids, Cα-methyl amino acids, Nα-methyl amino acids, and amino acid analogues in general. In addition, the amino acids can include Abu, 2-amino butyric acid; γ-Abu, 4-aminobutyric acid; ε-Ahx, 6-aminohexanoic acid; Aib, 2-amino-isobutyric acid; β-Ala, 3-aminopropionic acid; Orn, ornithine; Hyp, trans-hydroxyproline; Nle, norleucine; Nva, norvaline.

The three-letter indication “GLA” as used herein means 4-carboxyglutamic acid (γ-carboxyglutamate).

By “catalytically inactivated in the active site of the FVIIa polypeptide” is meant that a FVIIa inhibitor is bound to the FVIIa polypeptide and decreases or prevents the FVIIa-catalysed conversion of FX to FXa. A FVIIa inhibitor may be identified as a substance, which reduces the amidolytic activity by at least 50% at a concentration of the substance at 400 μM in the FVIIa amidolytic assay described by Persson et al. (Persson et al., J. Biol. Chem. 272: 19919-19924 (1997)). Preferred are substances reducing the amidolytic activity by at least 50% at a concentration of the substance at 300 μM; more preferred are substances reducing the amidolytic activity by at least 50% at a concentration of the substance at 200 μM.

The “FVIIa inhibitor” may be selected from any one of several groups of FVIIa directed inhibitors. Such inhibitors are broadly categorised for the purpose of the present invention into i) inhibitors which reversibly bind to FVIIa and are cleavable by FVIIa, ii) inhibitors which reversibly bind to FVIIa but cannot be cleaved, and iii) inhibitors which irreversibly bind to FVIIa. For a review of inhibitors of serine proteases see Proteinase Inhibitors (Research Monographs in cell and Tissue Physiology; v. 12) Elsevier Science Publishing Co., Inc., New York (1990).

The FVIIa inhibitor moiety may also be an irreversible FVIIa serine protease inhibitor. Such irreversible active site inhibitors generally form covalent bonds with the protease active site. Such irreversible inhibitors include, but are not limited to, general serine protease inhibitors such as peptide chloromethylketones (see, Williams et al., J. Biol. Chem. 264:7536-7540 (1989)) or peptidyl cloromethanes; azapeptides; acylating agents such as various guanidinobenzoate derivatives and the 3-alkoxy-4-chloroisocoumarins; sulphonyl fluorides such as phenylmethylsulphonylfluoride (PMSF); diisopropylfluorophosphate (DFP); tosylpropylchloromethyl ketone (TPCK); tosyllysylchloromethyl ketone (TLCK); nitrophenylsulphonates and related compounds; heterocyclic protease inhibitors such as isocoumarines, and coumarins.

Examples of peptidic irreversible FVIIa inhibitors include, but are not limited to,

Phe-Phe-Arg chloromethyl ketone, Phe-Phe-Arg chloromethylketone, D-Phe-Phe-Arg chloromethyl ketone, D-Phe-Phe-Arg chloromethylketone Phe-Pro-Arg chloromethylketone, D-Phe-Pro-Arg chloromethylketone, Phe-Pro-Arg chloromethylketone, D-Phe-Pro-Arg chloromethylketone, L-Glu-Gly-Arg chloromethylketone and D-Glu-Gly-Arg chloromethylketone.

Examples of FVIIa inhibitors also include benzoxazinones or heterocyclic analogues thereof such as described in PCT/DK99/00138.

Examples of other FVIIa inhibitors include, but are not limited to, small peptides such as for example Phe-Phe-Arg, D-Phe-Phe-Arg, Phe-Phe-Arg, D-Phe-Phe-Arg, Phe-Pro-Arg, D-Phe-Pro-Arg, Phe-Pro-Arg, D-Phe-Pro-Arg, L- and D-Glu-Gly-Arg; peptidomimetics; benzamidine systems; heterocyclic structures substituted with one or more amidino groups; aromatic or heteroaromatic systems substituted with one or more C(═NH)NHR groups in which R is H, C₁₋₃alkyl, OH or a group which is easily split of in vivo.

By “linker moiety” or “LM” is meant any biocompatible molecule functioning as a means to link the compound containing a radionuclide to the TF agonists and/or TF antagonist. The terms “linker”, linker part”, “linker part B”, “spacer” as used herein all refers to parts of the LM. The TF agonists and/or TF antagonist and the compound containing a radionuclide are linked to the molecular LM via a chemical bond, e.g. via an amide or peptide bond between an amino group of the LM and a carboxyl group, or its equivalent, of the TF agonists and/or TF antagonist and the compound containing a radionuclide, or vice versa. It is to be understood, that the LM may contain both covalent and non-covalent chemical bonds or mixtures thereof. The LM may comprise a plurality of carbon-carbon σ bonds having free rotation about their axes. Suitable LMs, or backbones, comprise group(s) such as, but are not limited to, peptides; polynucleotides; sacharides including monosaccharides, di- and oligosaccharides, cyclodextrins and dextran; polymers including polyethylene glycol, polypropylene glycol, polyvinyl alcohol, hydrocarbons, polyacrylates and amino-, hydroxy-, thio- or carboxy-functionalised silicones, other biocompatible material units; and combinations thereof. Such LM materials described above are widely commercially available or obtainable via synthetic organic methods commonly known to those skilled in the art. In a preferred embodiment of the invention the LM functions to release the compound containing a radionuclide of the compound having the formula A-(LM)-C. In one embodiment the LM functions to release the diagnostic compound following transfer to a reducing environment, e.g., cytoplasm or lysosomes. In another embodiment of the invention the LM functions to release the diagnostic compound following hydrolysis by specific hydrolases either inside the cell or on the cell surface, e.g. lysosomal proteases, such as Cathepsin B. In one embodiment of the invention the LM functions to release the diagnostic compound following an exogenous stimuli, e.g., light or other electromagnetic field radiation or ultrasound, e.g. high intensity focused ultrasound (HIFU).

The LM may, for example, comprise the following structures: straight or branched C₁₋₅₀-alkyl, straight or branched C₂₋₅₀-alkenyl, straight or branched C₂₋₅₀-alkynyl, a 1 to 50-membered straight or branched chain comprising carbon and at least one N, O or S atom in the chain, C₃₋₈cycloalkyl, a 3 to 8-membered cyclic ring comprising carbon and at least one N, O or S atom in the ring, aryl, heteroaryl, amino acid, the structures optionally substituted with one or more of the following groups: H, hydroxy, phenyl, phenoxy, benzyl, thienyl, oxo, amino, C₁₋₄-alkyl, —CONH₂, —CSNH₂, C₁₋₄ monoalkylamino, C₁₋₄ dialkylamino, acylamino, sulfonyl, carboxy, carboxamido, halogeno, C₁₋₆ alkoxy, C₁₋₆ alkylthio, trifluoroalkoxy, alkoxycarbonyl, haloalkyl. The LM may be straight chained or branched and may contain one or more double or triple bonds. The LM may contain one or more heteroatoms like N, O or S. It is to be understood, that the LM can comprise more than one class of the groups described above, as well as being able to comprise more than one member within a class. Where the LM comprises more than one class of group, such LM is preferably obtained by joining different units via their functional groups. Methods for forming such bonds involve standard organic synthesis and are well known to those of ordinary skill in the art.

By “combinations thereof” is meant that the LM can comprise more than one class of the groups described above, as well as being able to comprise more than one member within a class. Where the LM comprises more than one class of group, such LM is preferably obtained by joining different units via their functional groups. Methods for forming such bonds involve standard organic synthesis and are well known to those of ordinary skill in the art.

The LM can comprise functional groups, such as, for example hydroxy, oxo, amino, C₁₋₄ monoalkylamino, acylamino, sulfonyl, carboxy, carboxamido, halogeno, C₁₋₆ alkoxy, C₁₋₆ alkylthio, trifluoroalkoxy, alkoxycarbonyl, or haloalkyl groups. The LM can also comprise charged functional groups, such as for example, ammonium groups or carboxylate groups.

The charged functional groups can provide TF agonists and/or TF antagonists with sufficient solubility in aqueous or physiological systems, provide reactive sites for ionic bonding with other species, and enhance their avidity to other members of the TF/FVIIa/FXa complex. It is within the purview of one of skill in the art to select a particular acid, and concentration thereof, to confer optimal solubility and avidity properties to the TF agonists and/or TF antagonists. Preferably, the total amount of charged functional groups are minimised so as to maximise the TF agonists and/or TF antagonists specificity for TF sites, but not so as to significantly decrease solubility.

The terms “C₁₋₅₀-alkyl” or “C₁₋₅₀-alkanediyl” as used herein, refers to a straight or branched, saturated or unsaturated hydrocarbon chain having from one to 50 carbon atoms.

The terms “C₂₋₅₀-alkenyl” or “C₂₋₅₀-alkenediyl” as used herein, refers to an unsaturated branched or straight hydrocarbon chain having from 2 to 50 carbon atoms and at least one double bond.

The terms “C₂₋₅₀-alkynyl” or “C₂₋₅₀-alkynediyl” as used herein, refers to an unsaturated branched or straight hydrocarbon chain having from 2 to 50 carbon atoms and at least one triple bond. The C₁₋₅₀-alkyl residues include aliphatic hydrocarbon residues, unsaturated aliphatic hydrocarbon residues, alicyclic hydrocarbon residues. Examples of a C₁₋₅₀-alkyl within this definition include but are not limited to decanyl, hexadecanyl, octadecanyl, nonadecanyl, icosanyl, docosanyl, tetracosanyl, triacontanyl, decanediyl, hexadecanediyl, octadecanediyl, nonadecanediyl, icosanediyl, docosanediyl, tetracosanediyl, triacontanediyl,

The term C₃₋₈-cycloalkyl means an alicyclic hydrocarbon residue including saturated alicyclic hydrocarbon residues having 3 to 8 carbon atoms such as cyclopropyl, cyclobutyl, cyclopentyl, cyclohexyl; and C₅₋₆ unsaturated alicyclic hydrocarbon residues having 5 to 6 carbon atoms such as 1-cyclopentenyl, 2-cyclopentenyl, 3-cyclopentenyl, 1-cyclohexenyl, 2-cyclohexenyl, 3-cyclohexenyl.

The term “C₁₋₆-alkoxy” as used herein, alone or in combination, refers to a straight or branched monovalent substituent comprising a C₁₋₆-alkyl group linked through an ether oxygen having its free valence bond from the ether oxygen and having 1 to 6 carbon atoms e.g. methoxy, ethoxy, propoxy, isopropoxy, butoxy, pentoxy.

The term “C₁₋₆-alkylthio” as used herein, alone or in combination, refers to a straight or branched monovalent substituent comprising a C₁₋₆-alkyl group linked through an thioether sulfur atom having its free valence bond from the thioether sulfur and having 1 to 6 carbon atoms.

The terms “aryl” and “heteroaryl” as used herein refers to an aryl which can be optionally substituted or a heteroaryl which can be optionally substituted and includes phenyl, biphenyl, indene, fluorene, naphthyl (1-naphthyl, 2-naphthyl), anthracene (1-anthracenyl, 2-anthracenyl, 3-anthracenyl), thiophene (2-thienyl, 3-thienyl), furyl (2-furyl, 3-furyl), indolyl, oxadiazolyl, isoxazolyl, quinazolin, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyi, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl).

The invention also relates to partly or fully saturated analogues of the ring systems mentioned above.

The terms “C₁₋₄ monoalkylamino” and “C₁₋₄ dialkylamino” refer to an amino group having one or both of its hydrogens independently replaced by an alkyl group having 1 to 4 carbon atoms, alkyl being defined above, such as methylamino, dimethylamino, N-ethyl-N-methylamino, ethylamino, diethylamino, propylamino, dipropylamino, N-(n-butyl)-N-methylamino, n-butylamino, di(n-butyl)amino, sec-butylammino, t-butylamino, and the like.

The terms “acyl” or “carboxy” refer to a monovalent substituent comprising a C₁₋₆-alkyl group linked through a carbonyl group; such as e.g. acetyl, propionyl, butyryl, isobutyryl, pivaloyl, valeryl, and the like.

The term “acylamino” refers to the group C₁₋ₙ C(═O)NH—

The term “carboxamido” refers to the group —C(═O)NHC₁₋ₙ

The term “trifluoroalkoxy” refers to an C₁₋₆ alkoxy group as defined above having three of its hydrogen atoms bonded to one or more of the carbon atoms replaced by fluor atoms, such as (CF₃)O—, (CF₃)CH₂O—.

The term “alkoxycarbonyl” refers to the group —C(═O)(R) where R is an C₁₋₆ alkoxy group as defined above. The term “C₁₋₆-alkoxycarbonyl” as used herein refers to a monovalent substituent comprising a C₁₋₆-alkoxy group linked through a carbonyl group; such as e.g. methoxycarbonyl, carbethoxy, propoxycarbonyl, isopropoxycarbonyl, n-butoxycarbonyl, secbutoxycarbonyl, tert-butoxycarbonyl, 3-methylbutoxycarbonyl, n-hexoxycarbonyl and the like.

The term “leaving group” as used herein includes, but is not limited to, halogen, sulfonate or an acyl group. Suitable leaving groups will be known to a person skilled in the art.

“Halogen” refers to fluorine, chlorine, bromine, and iodine. “Halo” refers to fluoro, chloro, bromo and iodo.

“Optional” or “optionally” means that the subsequently described event or circumstances may or may not occur, and that the description includes instances where said event or circumstance occur and instances in which is does not. For example, “aryl . . . optionally substituted” means that the aryl may or may not be substituted and that the description includes both unsubstituted aryls and aryls wherein there is substitution

In one embodiment of the invention, LM comprises a FVIIa inhibitor. It is to be understood, that the FVIIa inhibitor is used to conjugate the compound containing a radionuclide via LM comprising the FVIIa inhibitor into the active site of a FVIIa polypeptide.

The compound containing a radionuclide linker moiety conjugates C-(LM) comprising a FVIIa inhibitor to be used in the preparation of a TF agonists and/or TF antagonist may be prepared by the following methods. In the following methods the FVIIa inhibitor is designated the letter F. The compound containing a radionuclide C is designated the letter C. Linker part B refers to other linker part of the LM.

Method 1.

LM comprising FVIIa inhibitors is prepared by reacting F-B-X, in which X is a functional group capable of reacting with structures C-Y, in which Y is a functional group, by means of normal coupling reactions using coupling reagents known by the person skilled in the art.

Method 2.

LM comprising FVIIa inhibitors may be prepared by reaction between F-B-Z, in which Z is a leaving group and C-W in which W is a nucleophile. Examples of leaving groups are halogens, sulfonates, phosphonates. Examples of nucleophiles are hydroxy, amino, N-substituted amino, and carbanions.

Method 3.

LM comprising FVIIa inhibitors may be prepared by reaction between C-B-Z, in which Z is a leaving group, and F-W, in which W is a nucleophile. Examples of leaving groups are halogens, sulfonates, phosphonates. Examples of nucleophiles are hydroxy, amino, N-substituted amino, and carbanions.

Method 4.

The linker part B can be reacted with structures F and C connected to a solid phase surface using methods well known in the art.

Method 5.

The compound containing a radionuclide linker moiety conjugates C-(LM) comprising a FVIIa inhibitor may be prepared by a sequence of reactions through which F or C firstly are reacted with the activated linker moiety forming F-B, respectively C-B moieties and subsequently the formed product is reacted with C, respectively F moiety. The actual bond formation taking place through reaction on functional groups or derivatives or leaving groups/nucleophiles as described under methods 1-3.

The reaction can be carried out in solution phase or on a solid phase support using procedures known by the person skilled in the art.

In the present specification, amino acids are represented using abbreviations, as indicated in table 1, approved by IUPAC-IUB Commission on Biochemical Nomenclature (CBN). Amino acid and the like having isomers represented by name or the following abbreviations are in natural L-form unless otherwise indicated. Further, the left and right ends of an amino acid sequence of a peptide are, respectively, the N- and C-termini unless otherwise specified.

TABLE 1
Abbreviations for amino acids:

| Amino acid | Tree-letter code | One-letter code |
|---|---|---|
| Glycine | Gly | G |
| Proline | Pro | P |
| Alanine | Ala | A |
| Valine | Val | V |
| Leucine | Leu | L |
| Isoleucine | Ile | I |
| Methionine | Met | M |
| Cysteine | Cys | C |
| Phenylalanine | Phe | F |
| Tyrosine | Tyr | Y |
| Tryptophan | Trp | W |
| Histidine | His | H |
| Lysine | Lys | K |
| Argmine | Arg | R |
| Glutamine | Gln | Q |
| Asparagine | Asn | N |
| Glutamic Acid | Glu | E |
| Aspartic Acid | Asp | D |
| Serine | Ser | S |
| Threonine | Thr | T |

The invention also relates to a method of preparing TF agonists and/or TF antagonists as mentioned above. The TF agonists and/or TF antagonist may be produced by recombinant DNA techniques. To this end, DNA sequences encoding human FVIIa may be isolated by preparing a genomic or cDNA library and screening for DNA sequences coding for all or part of the protein by hybridization using synthetic oligonucleotide probes in accordance with standard techniques (cf. Sambrook et al., Molecular Cloning: A Laboratory Manual, Cold Spring Harbor Laboratory, Cold Spring Harbor, N.Y., 1989). For the present purpose, the DNA sequence encoding the protein is preferably of human origin, i.e. derived from a human genomic DNA or cDNA library.

The DNA sequences encoding the human FVIIa polypeptides may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesized, e.g. in an automatic DNA synthesizer, purified, annealed, ligated and cloned in suitable vectors.

The DNA sequences may also be prepared by polymerase chain reaction using specific primers, for instance as described in U.S. Pat. No. 4,683,202, Saiki et al., Science 239 (1988), 487-491, or Sambrook et al., supra.

The DNA sequences encoding the human FVIIa polypeptides are usually inserted into a recombinant vector which may be any vector, which may conveniently be subjected to recombinant DNA procedures, and the choice of vector will often depend on the host cell into which it is to be introduced. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the human FVIIa polypeptides is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the polypeptide.

The promoter may be any DNA sequence, which shows transcriptional activity in the host cell of choice and may be derived from genes encoding proteins either homologous or heterologous to the host cell.

Examples of suitable promoters for directing the transcription of the DNA encoding the human FVIIa polypeptide in mammalian cells are the SV40 promoter (Subramani et al., Mol. Cell Biol. 1 (1981), 854-864), the MT-1 (metallothionein gene) promoter (Palmiter et al., Science 222 (1983), 809-814), the CMV promoter (Boshart et al., Cell 41:521-530, 1985) or the adenovirus 2 major late promoter (Kaufman and Sharp, Mol. Cell. Biol, 2:1304-1319, 1982).

An example of a suitable promoter for use in insect cells is the polyhedrin promoter (U.S. Pat. No. 4,745,051; Vasuvedan et al., FEBS Lett. 311, (1992)7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. No. 5,155,037; U.S. Pat. No. 5,162,222), or the baculovirus 39K delayed-early gene promoter (U.S. Pat. No. 5,155,037; U.S. Pat. No. 5,162,222).

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4c (Russell et al., Nature 304 (1983), 652-654) promoters.

Examples of suitable promoters for use in filamentous fungus host cells are, for instance, the ADH3 promoter (McKnight et al., The EMBO J. 4 (1985), 2093-2099) or the tpiA promoter. Examples of other useful promoters are those derived from the gene encoding A. oryzae TAKA amylase, Rhizomucor miehei aspartic proteinase, A. niger neutral α-amylase, A. niger acid stable α-amylase, A. niger or A. awamori glucoamylase (gluA), Rhizomucor miehei lipase, A. oryzae alkaline protease, A. oryzae triose phosphate isomerase or A. nidulans acetamidase. Preferred are the TAKA-amylase and gluA promoters. Suitable promoters are mentioned in, e.g. EP 238 023 and EP 383 779.

The DNA sequences encoding the human FVIIa polypeptides may also, if necessary, be operably connected to a suitable terminator, such as the human growth hormone terminator (Palmiter et al., Science 222, 1983, pp. 809-814) or the TPI1 (Alber and Kawasaki, J. Mol. Appl. Gen. 1, 1982, pp. 419-434) or ADH3 (McKnight et al., The EMBO J. 4, 1985, pp. 2093-2099) terminators. The vector may also contain a set of RNA splice sites located downstream from the promoter and upstream from the insertion site for the FVIIa sequence itself. Preferred RNA splice sites may be obtained from adenovirus and/or immunoglobulin genes. Also contained in the expression vectors is a polyadenylation signal located downstream of the insertion site. Particularly preferred polyadenylation signals include the early or late polyadenylation signal from SV40 (Kaufman and Sharp, ibid.), the polyadenylation signal from the adenovirus 5 Elb region, the human growth hormone gene terminator (DeNoto et al. Nuc. Acids Res. 9:3719-3730, 1981) or the polyadenylation signal from the human FVII gene or the bovine FVII gene. The expression vectors may also include a noncoding viral leader sequence, such as the adenovirus 2 tripartite leader, located between the promoter and the RNA splice sites; and enhancer sequences, such as the SV40 enhancer.

The recombinant vector may further comprise a DNA sequence enabling the vector to replicate in the host cell in question. An example of such a sequence (when the host cell is a mammalian cell) is the SV40 origin of replication.

When the host cell is a yeast cell, suitable sequences enabling the vector to replicate are the yeast plasmid 2μ replication genes REP 1-3 and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g. ampicillin, kanamycin, tetracyclin, chloramphenicol, neomycin, hygromycin or methotrexate. For filamentous fungi, selectable markers include amdS, pyrG, argB, niaD or sC.

To direct the human FVIIa polypeptides of the present invention into the secretory pathway of the host cells, a secretory signal sequence (also known as a leader sequence, prepro sequence or pre sequence) may be provided in the recombinant vector. The secretory signal sequence is joined to the DNA sequences encoding the human FVIIa polypeptides in the correct reading frame. Secretory signal sequences are commonly positioned 5′ to the DNA sequence encoding the peptide. The secretory signal sequence may be that, normally associated with the protein or may be from a gene encoding another secreted protein.

For secretion from yeast cells, the secretory signal sequence may encode any signal peptide, which ensures efficient direction of the expressed human FVIIa polypeptides into the secretory pathway of the cell. The signal peptide may be naturally occurring signal peptide, or a functional part thereof, or it may be a synthetic peptide. Suitable signal peptides have been found to be the α-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137).

For efficient secretion in yeast, a sequence encoding a leader peptide may also be inserted downstream of the signal sequence and upstream of the DNA sequence encoding the human FVIIa polypeptides. The function of the leader peptide is to allow the expressed peptide to be directed from the endoplasmic reticulum to the Golgi apparatus and further to a secretory vesicle for secretion into the culture medium (i.e. exportation of the human FVIIa polypeptides across the cell wall or at least through the cellular membrane into the periplasmic space of the yeast cell). The leader peptide may be the yeast alpha-factor leader (the use of which is described in e.g. U.S. Pat. No. 4,546,082, U.S. Pat. No. 4,870,008, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase. Suitable signal peptides are disclosed in, e.g. EP 238 023 and EP 215 594.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

The procedures used to ligate the DNA sequences coding for the human FVIIa polypeptides, the promoter and optionally the terminator and/or secretory signal sequence, respectively, and to insert them into suitable vectors containing the information necessary for replication, are well known to persons skilled in the art (cf., for instance, Sambrook et al., Molecular Cloning: A Laboratory Manual, Cold Spring Harbor, N.Y., 1989).

Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Graham and van der Eb, Virology 52 (1973), 456; and Neumann et al., EMBO J. 1 (1982), 841-845.

Selectable markers may be introduced into the cell on a separate plasmid at the same time as the gene of interest, or they may be introduced on the same plasmid. If on the same plasmid, the selectable marker and the gene of interest may be under the control of different promoters or the same promoter, the latter arrangement producing a dicistronic message. Constructs of this type are known in the art (for example, Levinson and Simonsen, U.S. Pat. No. 4,713,339). It may also be advantageous to add additional DNA, known as “carrier DNA,” to the mixture that is introduced into the cells.

After the cells have taken up the DNA, they are grown in an appropriate growth medium, typically 1-2 days, to begin expressing the gene of interest. As used herein the term “appropriate growth medium” means a medium containing nutrients and other components required for the growth of cells and the expression of the human FVIIa polypeptides of interest. Media generally include a carbon source, a nitrogen source, essential amino acids, essential sugars, vitamins, salts, phospholipids, protein and growth factors. For production of gammacarboxylated proteins, the medium will contain vitamin K, preferably at a concentration of about 0.1 μg/ml to about 5 μg/ml. Drug selection is then applied to select for the growth of cells that are expressing the selectable marker in a stable fashion. For cells that have been transfected with an amplifiable selectable marker the drug concentration may be increased to select for an increased copy number of the cloned sequences, thereby increasing expression levels. Clones of stably transfected cells are then screened for expression of the human FVIIa polypeptide of interest.

The host cell into which the DNA sequences encoding the human FVIIa polypeptides is introduced may be any cell, which is capable of producing the posttranslational modified human FVIIa polypeptides and includes yeast, fungi and higher eukaryotic cells.

Examples of mammalian cell lines for use in the present invention are the COS-1 (ATCC CRL 1650), baby hamster kidney (BHK) and 293 (ATCC CRL 1573; Graham et al., J. Gen. Virol. 36:59-72, 1977) cell lines. A preferred BHK cell line is the tk⁻ ts13 BHK cell line (Waechter and Baserga, Proc. Natl. Acad. Sci. USA 79:1106-1110, 1982, incorporated herein by reference), hereinafter referred to as BHK 570 cells. The BHK 570 cell line has been deposited with the American Type Culture Collection, 12301 Parklawn Dr., Rockville, Md. 20852, under ATCC accession number CRL 10314. A tk⁻ ts13 BHK cell line is also available from the ATCC under accession number CRL 1632. In addition, a number of other cell lines may be used within the present invention, including Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1), CHO (ATCC CCL 61) and DUKX cells (Urlaub and Chasin, Proc. Natl. Acad. Sci. USA 77:4216-4220, 1980).

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous polypeptides there from are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequences encoding the human FVIIa polypeptides may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 1986, pp. 3459-3465; U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277, EP 238 023, EP 184 438 The transformation of F. oxysporum may, for instance, be carried out as described by Malardier et al., 1989, Gene 78: 147-156. The transformation of Trichoderma spp. may be performed for instance as described in EP 244 234.

When a filamentous fungus is used as the host cell, it may be transformed with the DNA construct of the invention, conveniently by integrating the DNA construct in the host chromosome to obtain a recombinant host cell. This integration is generally considered to be an advantage as the DNA sequence is more likely to be stably maintained in the cell. Integration of the DNA constructs into the host chromosome may be performed according to conventional methods, e.g. by homologous or heterologous recombination.

Transformation of insect cells and production of heterologous polypeptides therein may be performed as described in U.S. Pat. No. 4,745,051; U.S. Pat. No. 4,879,236; U.S. Pat. Nos. 5,155,037; 5,162,222; EP 397,485) all of which are incorporated herein by reference. The insect cell line used as the host may suitably be a Lepidoptera cell line, such as Spodoptera frugiperda cells or Trichoplusia ni cells (cf. U.S. Pat. No. 5,077,214). Culture conditions may suitably be as described in, for instance, WO 89/01029 or WO 89/01028, or any of the aforementioned references.

The transformed or transfected host cell described above is then cultured in a suitable nutrient medium under conditions permitting expression of the human FVIIa polypeptide after which all or part of the resulting peptide may be recovered from the culture. The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The human FVIIa polypeptide produced by the cells may then be recovered from the culture medium by conventional procedures including separating the host cells from the medium by centrifugation or filtration, precipitating the proteinaqueous components of the supernatant or filtrate by means of a salt, e.g. ammonium sulphate, purification by a variety of chromatographic procedures, e.g. ion exchange chromatography, gel filtration chromatography, affinity chromatography, or the like, dependent on the type of polypeptide in question.

For the preparation of recombinant human FVIIa polypeptides, a cloned wild-type FVIIa DNA sequence is used. This sequence may be modified to encode a desired FVIIa variant. The complete nucleotide and amino acid sequences for human FVIIa are known. See U.S. Pat. No. 4,784,950, which is incorporated herein by reference, where the cloning and expression of recombinant human FVIIa is described. The bovine FVIIa sequence is described in Takeya et al., J. Biol. Chem, 263:14868-14872 (1988), which is incorporated by reference herein.

The amino acid sequence alterations may be accomplished by a variety of techniques. Modification of the DNA sequence may be by site-specific mutagenesis. Techniques for site-specific mutagenesis are well known in the art and are described by, for example, Zoller and Smith (DNA 3:479-488, 1984). Thus, using the nucleotide and amino acid sequences of FVII, one may introduce the alterations of choice.

DNA sequences for use within the present invention will typically encode a pre-pro peptide at the amino-terminus of the FVIIa protein to obtain proper post-translational processing (e.g. gamma-carboxylation of glutamic acid residues) and secretion from the host cell. The pre-pro peptide may be that of FVIIa or another vitamin K-dependent plasma protein, such as factor IX, factor X, prothrombin, protein C or protein S. As will be appreciated by those skilled in the art, additional modifications can be made in the amino acid sequence of FVIIa where those modifications do not significantly impair the ability of the protein to act as a coagulation factor. For example, FVIIa in the catalytic triad can also be modified in the activation cleavage site to inhibit the conversion of zymogen FVII into its activated two-chain form, as generally described in U.S. Pat. No. 5,288,629, incorporated herein by reference.

Within the present invention, transgenic animal technology may be employed to produce the human FVIIa polypeptide. It is preferred to produce the proteins within the mammary glands of a host female mammal. Expression in the mammary gland and subsequent secretion of the protein of interest into the milk overcomes many difficulties encountered in isolating proteins from other sources. Milk is readily collected, available in large quantities, and well characterized biochemically. Furthermore, the major milk proteins are present in milk at high concentrations (typically from about 1 to 15 g/l). From a commercial point of view, it is clearly preferable to use as the host a species that has a large milk yield. While smaller animals such as mice and rats can be used (and are preferred at the proof of principle stage), within the present invention it is preferred to use livestock mammals including, but not limited to, pigs, goats, sheep and cattle. Sheep are particularly preferred due to such factors as the previous history of transgenesis in this species, milk yield, cost and the ready availability of equipment for collecting sheep milk. See WIPO Publication WO 88/00239 for a comparison of factors influencing the choice of host species. It is generally desirable to select a breed of host animal that has been bred for dairy use, such as East Friesland sheep, or to introduce dairy stock by breeding of the transgenic line at a later date. In any event, animals of known, good health status should be used.

To obtain expression in the mammary gland, a transcription promoter from a milk protein gene is used. Milk protein genes include those genes encoding caseins (see U.S. Pat. No. 5,304,489, incorporated herein by reference), beta-lactoglobulin, alpha-lactalbumin, and whey acidic protein. The beta-lactoglobulin (BLG) promoter is preferred. In the case of the ovine beta-lactoglobulin gene, a region of at least the proximal 406 bp of 5′ flanking sequence of the gene will generally be used, although larger portions of the 5′ flanking sequence, up to about 5 kbp, are preferred, such as about 4.25 kbp DNA segment encompassing the 5′ flanking promoter and non-coding portion of the beta-lactoglobulin gene. See Whitelaw et al., Biochem J. 286: 31-39 (1992). Similar fragments of promoter DNA from other species are also suitable.

Other regions of the beta-lactoglobulin gene may also be incorporated in constructs, as may genomic regions of the gene to be expressed. It is generally accepted in the art that constructs lacking introns, for example, express poorly in comparison with those that contain such DNA sequences (see Brinster et al., Proc. Natl. Acad. Sci. USA 85: 836-840 (1988); Palmiter et al., Proc. Natl. Acad. Sci. USA 88: 478-482 (1991); Whitelaw et al., Transgenic Res. 1: 3-13 (1991); WO 89/01343; and WO 91/02318, each of which is incorporated herein by reference). In this regard, it is generally preferred, where possible, to use genomic sequences containing all or some of the native introns of a gene encoding the protein or polypeptide of interest, thus the further inclusion of at least some introns from, e.g., the beta-lactoglobulin gene, is preferred. One such region is a DNA segment which provides for intron splicing and RNA polyadenylation from the 3′ non-coding region of the ovine beta-lactoglobulin gene. When substituted for the natural 3′ non-coding sequences of a gene, this ovine beta-lactoglobulin segment can both enhance and stabilize expression levels of the protein or polypeptide of interest. Within other embodiments, the region surrounding the initiation ATG of the sequence encoding the human FVIIa polypeptide is replaced with corresponding sequences from a milk specific protein gene. Such replacement provides a putative tissue-specific initiation environment to enhance expression. It is convenient to replace the entire pre-pro sequence of the human FVIIa polypeptide and 5′ non-coding sequences with those of, for example, the BLG gene, although smaller regions may be replaced.

For expression of a human FVIIa polypeptide in transgenic animals, a DNA segment encoding the human FVIIa polypeptide is operably linked to additional DNA segments required for its expression to produce expression units. Such additional segments include the abovementioned promoter, as well as sequences which provide for termination of transcription and polyadenylation of mRNA. The expression units will further include a DNA segment encoding a secretory signal sequence operably linked to the segment encoding the human FVIIa polypeptide. The secretory signal sequence may be a native secretory signal sequence of the human FVIIa polypeptide or may be that of another protein, such as a milk protein. See, for example, von Heinje, Nuc. Acids Res. 14: 4683-4690 (1986); and Meade et al., U.S. Pat. No. 4,873,316, which are incorporated herein by reference.

Construction of expression units for use in transgenic animals is conveniently carried out by inserting a sequence encoding the human FVIIa polypeptide into a plasmid or phage vector containing the additional DNA segments, although the expression unit may be constructed by essentially any sequence of ligations. It is particularly convenient to provide a vector containing a DNA segment encoding a milk protein and to replace the coding sequence for the milk protein with that of the human FVIIa polypeptide, thereby creating a gene fusion that includes the expression control sequences of the milk protein gene. In any event, cloning of the expression units in plasmids or other vectors facilitates the amplification of the human FVIIa polypeptide. Amplification is conveniently carried out in bacterial (e.g. E. coli) host cells, thus the vectors will typically include an origin of replication and a selectable marker functional in bacterial host cells.

The expression unit is then introduced into fertilized eggs (including early-stage embryos) of the chosen host species. Introduction of heterologous DNA can be accomplished by one of several routes, including microinjection (e.g. U.S. Pat. No. 4,873,191), retroviral infection (Jaenisch, Science 240: 1468-1474 (1988)) or site-directed integration using embryonic stem (ES) cells (reviewed by Bradley et al., Bio/Technology 10: 534-539 (1992)). The eggs are then implanted into the oviducts or uteri of pseudopregnant females and allowed to develop. Offspring carrying the introduced DNA in their germ line can pass the DNA on to their progeny in the normal, Mendelian fashion, allowing the development of transgenic herds.

General procedures for producing transgenic animals are known in the art. See, for example, Hogan et al., Manipulating the Mouse Embryo: A Laboratory Manual, Cold Spring Harbor Laboratory, 1986; Simons et al., Bio/Technology 6: 179-183 (1988); Wall et al., Biol. Reprod. 32: 645-651 (1985); Buhler et al., Bio/Technology 8: 140-143 (1990); Ebert et al., Bio/Technology 9: 835-838 (1991); Krimpenfort et al., Bio/Technology 9: 844-847 (1991); Wall et al., J. Cell. Biochem. 49:113-120 (1992); U.S. Pat. Nos. 4,873,191 and 4,873,316; WIPO publications WO 88/00239, WO 90/05188, WO 92/11757; and GB 87/00458, which are incorporated herein by reference. Techniques for introducing foreign DNA sequences into mammals and their germ cells were originally developed in the mouse. See, e.g., Gordon et al., Proc. Natl. Acad. Sci. USA 77: 7380-7384 (1980); Gordon and Ruddle, Science 214: 1244-1246 (1981); Palmiter and Brinster, Cell 41: 343-345 (1985); and Brinster et al., Proc. Natl. Acad. Sci. USA 82: 4438-4442 (1985). These techniques were subsequently adapted for use with larger animals, including livestock species (see e.g., WIPO publications WO 88/00239, WO 90/05188, and WO 92/11757; and Simons et al., Bio/Technology 6: 179-183 (1988). To summarize, in the most efficient route used to date in the generation of transgenic mice or livestock, several hundred linear molecules of the DNA of interest are injected into one of the pro-nuclei of a fertilized egg according to established techniques. Injection of DNA into the cytoplasm of a zygote can also be employed. Production in transgenic plants may also be employed. Expression may be generalized or directed to a particular organ, such as a tuber. See, Hiatt, Nature 344:469-479 (1990); Edelbaum et al., J. Interferon Res. 12:449-453 (1992); Sijmons et al., Bio/Technology 8:217-221 (1990); and European Patent Office Publication EP 255,378.

FVIIa produced according to the present invention may be purified by affinity chromatography on an anti-FVII antibody column. It is preferred that the immunoadsorption column comprise a high-specificity monoclonal antibody. The use of calcium-dependent monoclonal antibodies, as described by Wakabayashi et al., J. Biol. Chem, 261:11097-11108, (1986) and Thim et al., Biochem. 27: 7785-7793, (1988), incorporated by reference herein, is particularly preferred. Additional purification may be achieved by conventional chemical purification means, such as high performance liquid chromatography. Other methods of purification, including barium citrate precipitation, are known in the art, and may be applied to the purification of the FVIIa described herein (see, generally, Scopes, R., Protein Purification, Springer-Verlag, N.Y., 1982). Substantially pure FVIIa of at least about 90 to 95% homogeneity is preferred, and 98 to 99% or more homogeneity most preferred, for pharmaceutical uses. Once purified, partially or to homogeneity as desired, the FVIIa may then be used therapeutically.

Conversion of single-chain FVII to active two-chain FVIIa may be achieved using factor XIIa as described by Hedner and Kisiel (1983, J. Clin. Invest. 71: 1836-1841), or with other proteases having trypsin-like specificity (Kisiel and Fujikawa, Behring Inst Mitt. 73: 29-42, 1983). Alternatively FVII may be autoactivated by passing it through an ion-exchange chromatography column, such as mono Q.RTM. (Pharmacia Fire Chemicals) or the like (Bjoern et al., 1986, Research Disclosures 269:564-565). The FVIIa molecules of the present invention and pharmaceutical compositions thereof are particularly useful for administration to humans to treat a variety of conditions involving intravascular coagulation.

The compounds of the present invention may have one or more asymmetric centers and it is intended that stereoisomers (optical isomers), as separated, pure or partially purified stereoisomers or racemic mixtures thereof are included in the scope of the invention.

Within the present invention, the TF agonists and/or TF antagonist may be prepared in the form of pharmaceutically acceptable salts, especially acid-addition salts, including salts of organic acids and mineral acids. Examples of such salts include salts of organic acids such as formic acid, fumaric acid, acetic acid, propionic acid, glycolic acid, lactic acid, pyruvic acid, oxalic acid, succinic acid, malic acid, tartaric acid, citric acid, benzoic acid, salicylic acid and the like. Suitable inorganic acid-addition salts include salts of hydrochloric, hydrobromic, sulphuric and phosphoric acids and the like. Further examples of pharmaceutically acceptable inorganic or organic acid addition salts include the pharmaceutically acceptable salts listed in Journal of Pharmaceutical Science, 66, 2 (1977) which are known to the skilled artisan.

Also intended as pharmaceutically acceptable acid addition salts are the hydrates which the present compounds are able to form.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent.

The compounds of this invention may form solvates with standard low molecular weight solvents using methods known to the skilled artisan.

The TF agonists and/or TF antagonist of the invention are useful for the preparation of a pharmaceutical composition for the diagnosing, treatment of or prophylaxis of thrombotic or coagulopathic related diseases or disorders including vascular diseases and inflammatory responses. Such diseases and responses include, but are not limited to bleedings, deep venous thrombosis, arterial thrombosis, post surgical thrombosis, coronary artery bypass graft (CABG), percutaneous transdermal coronary angioplastry (PTCA), stroke, tumour metastasis, inflammation, septic chock, hypotension, ARDS, pulmonary embolism, disseminated intravascular coagulation (DIC), vascular restenosis, platelet deposition, myocardial infarction, angiogenesis, or the prophylactic treatment of mammals with atherosclerotic vessels at risk for thrombosis.

The TF agonists and/or TF antagonist may be administered in pharmaceutically acceptable acid addition salt form or, where appropriate, as a alkali metal or alkaline earth metal or lower alkylammonium salt. Such salt forms are believed to exhibit approximately the same order of activity as the free base forms.

Apart from the pharmaceutical use of the compounds, they may be useful in vitro tools for investigating the inhibition of FVIIa, FXa or TF/FVIIa/FXa activity.

Pharmaceutical Compositions

Another object of the present invention is to provide a pharmaceutical formulation comprising a TF binding conjugate which has a pH from 2.0 to 10.0. The formulation may further comprise a buffer system, preservative(s), isotonicity agent(s), chelating agent(s), stabilizers and surfactants. In one embodiment of the invention the pharmaceutical formulation is an aqueous formulation, i.e. formulation comprising water. Such formulation is typically a solution or a suspension. In a further embodiment of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment the pharmaceutical formulation is a freeze-dried formulation, whereto the physician or the patient adds solvents and/or diluents prior to use.

In another embodiment the pharmaceutical formulation is a dried formulation (e.g. freeze-dried or spray-dried) ready for use without any prior dissolution.

In a further aspect the invention relates to a pharmaceutical formulation comprising an aqueous solution of a TF binding conjugate, and a buffer, wherein said formulation has a pH from about 2.0 to about 10.0.

In a another embodiment of the invention the pH of the formulation is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0.

In a further embodiment of the invention the buffer is selected from the group consisting of sodium acetate, sodium carbonate, citrate, glycylglycine, histidine, glycine, lysine, arginin, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment of the invention the formulation further comprises a pharmaceutically acceptable preservative. In a further embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 19^{th} edition, 1995.

In a further embodiment of the invention the formulation further comprises an isotonic agent. In a further embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one —OH group and includes, for example, mannitol, sorbitol, inositol, galacititol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 19^{th} edition, 1995.

In a further embodiment of the invention the formulation further comprises a chelating agent. In a further embodiment of the invention the chelating agent is selected from salts of ethylenediaminetetraacetic acid (EDTA), citric acid, and aspartic acid, and mixtures thereof. In a further embodiment of the invention the chelating agent is present in a concentration from 0.1 mg/ml to 5 mg/ml. In a further embodiment of the invention the chelating agent is present in a concentration from 0.1 mg/ml to 2 mg/ml. In a further embodiment of the invention the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 19^{th} edition, 1995.

In a further embodiment of the invention the formulation further comprises a stabiliser. The use of a stabilizer in pharmaceutical compositions is well-known to the skilled person. For convenience reference is made to Remington: The Science and Practice of Pharmacy, 19^{th} edition, 1995.

More particularly, compositions of the invention are stabilized liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L, D, or DL isomer) of a particular amino acid (e.g. glycine, methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, ornithine and N-monoethyl L-arginine, suitable methionine analogues include ethionine and buthionine and suitable cystein analogues include S-methyl-L cystein. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further embodiment of the invention methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidized species over time. Inhibiting methionine oxidation results in greater retention of the polypeptide in its proper molecular form. Any stereoisomer of methionine (L, D, or DL isomer) or combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In a further embodiment of the invention the formulation further comprises a stabiliser selected from the group of high molecular weight polymers or low molecular compounds. In a further embodiment of the invention the stabilizer is selected from polyethylene glycol (e.g. PEG 3350), polyvinylalcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a nonionic surfactant, which protects the polypeptide against aggregation associated with freeze-thawing or mechanical shearing.

In a further embodiment of the invention the formulation further comprises a surfactant. In a further embodiment of the invention the surfactant is selected from a detergent, ethoxylated castor oil, polyglycolyzed glycerides, acetylated monoglycerides, sorbitan fatty acid esters, polyoxypropylene-polyoxyethylene block polymers (e.g. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lecitins and phospholipids (e.g. phosphatidyl serine, phosphatidyl choline, phosphatidyl ethanolamine, phosphatidyl inositol, diphosphatidyl glycerol and sphingomyelin), derivates of phospholipids (e.g. dipalmitoyl phosphatidic acid) and lysophospholipids (e.g. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (e.g. cephalins), glyceroglycolipids (e.g. galactopyransoide), sphingoglycolipids (e.g. ceramides, gangliosides), dodecylphosphocholine, hen egg lysolecithin, fusidic acid derivatives-(e.g. sodium tauro-dihydrofusidate etc.), long-chain fatty acids and salts thereof C6-C12 (e.g. oleic acid and caprylic acid), acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulfate or sodium lauryl sulfate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quarternary ammonium bases) (e.g. cetyl-trimethylammonium bromide, cetylpyridinium chloride), non-ionic surfactants (e.g. Dodecyl β-D-glucopyranoside), poloxamines (e.g. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 19^{th} edition, 1995.

It is possible that other ingredients may be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatin or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Pharmaceutical compositions containing a TF binding conjugate according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for ex-ample in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the TF binding conjugate, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Examples of solid carriers are lactose, terra alba, sucrose, talc, gelatine, agar, pectin, acacia, magnesium stearate and stearic acid. Examples of liquid carriers are syrup, peanut oil, olive oil and water. Similarly, the carrier or diluent may include any time delay material known to the art, such as glyceryl monostearate or glyceryl distearate, alone or mixed with a wax. The formulations may also include wetting agents, emulsifying and suspending agents, preserving agents, sweetening agents or flavouring agents. The formulations of the invention may be formulated so as to provide quick, sustained, or delayed release of the active ingredient after administration to the patient by employing procedures well known in the art.

Compositions of the current invention are useful in the formulation of solids, semisolids, powder and solutions for pulmonary administration of the TF binding conjugate, using, for example a metered dose inhaler, dry powder inhaler and a nebulizer, all being devices well known to those skilled in the art.

Compositions of the current invention are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, co-cystallization, precipitation, co-precipitation, emulsification, dispersion, high pressure homogenization, encapsulation, spray drying, microencapsulation, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the TF binding conjugate in the form of a nasal or pulmonal spray. As a still further option, the pharmaceutical compositions containing the TF binding conjugate of the invention can also be adapted to transdermal administration, e.g. by needle-free injection or from a patch, optionally an iontophoretic patch, or transmucosal, e.g. buccal, administration.

The term “stabilized formulation” refers to a formulation with increased physical stability, increased chemical stability or increased physical and chemical stability.

The term “physical stability” of the protein formulation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to protein aggregation, when it shows visual turbidity in daylight. Alternatively, the turbidity of the formulation can be evaluated by simple turbidity measurements well-known to the skilled person. Physical stability of the aqueous protein formulations can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as antrhacene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein formulation as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein formulation as well-known by the person skilled in the art. Most proteins are prone to deamidation, a process in which the side chain amide group in glutaminyl or asparaginyl residues is hydrolysed to form a free carboxylic acid. Other degradations pathways involves form ation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized formulation” refers to a formulation with increased physical stability, increased chemical stability or increased physical and chemical stability. In general, a formulation must be stable during use and storage (in compliance with recommended use and storage conditions) until the expiration date is reached.

In one embodiment of the invention the pharmaceutical formulation comprising the TF binding conjugate is stable for more than 6 weeks of usage and for more than 3 years of storage.

In another embodiment of the invention the pharmaceutical formulation comprising the TF binding conjugate is stable for more than 4 weeks of usage and for more than 3 years of storage.

In a further embodiment of the invention the pharmaceutical formulation comprising the TF binding conjugate is stable for more than 4 weeks of usage and for more than two years of storage.

In an even further embodiment of the invention the pharmaceutical formulation comprising the TF binding conjugate is stable for more than 2 weeks of usage and for more than two years of storage.

Optionally, the pharmaceutical composition of the invention may comprise a TF antagonist in combination with one or more other compounds exhibiting anticoagulant activity, e.g., platelet aggregation inhibitor.

As used herein, “pharmaceutically acceptable carriers” also encompasses any and all solvents, dispersion media, coatings, antifungal agents, and the like. Except insofar as any conventional medium is incompatible with the active ingredient and its intended use, its use in the compositions of the present invention is contemplated.

The pharmaceutical compositions can be sterilised and mixed, if desired, with auxiliary agents, emulsifiers, salt for influencing osmotic pressure, buffers and/or colouring substances and the like, which do not deleteriously react with the active compounds.

The route of administration may be any route, which effectively transports the active compound to the appropriate or desired site of action, such as oral or parenteral, e.g., rectal, transdermal, subcutaneous, intranasal, intramuscular, topical, intravenous, intraurethral, ophthalmic solution or an ointment, the oral route being preferred.

If a solid carrier for oral administration is used, the preparation can be tabletted, placed in a hard gelatine capsule in powder or pellet form or it can be in the form of a troche or lozenge. The amount of solid carrier may vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

For nasal administration, the preparation may contain a compound of formula (I) dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet, which may be prepared by conventional tabletting techniques, contains

Core:

| | |
|---|---|
| Active compound (as free compound | 10 mg |
| or salt thereof) | |
| Colloidal silicon dioxide (Areosil ®) | 1.5 mg |
| Cellulose, microcryst. (Avicel ®) | 70 mg |
| Modified cellulose gum (Ac-Di-Sol ®) | 7.5 mg |
| Magnesium stearate | |

Coating:

| | |
|---|---|
| HPMC | approx. 9 mg |
| *Mywacett ® 9-40 T | approx. 0.9 mg |

*Acylated monoglyceride used as plasticizer for film coating.

The compounds of the invention may be administered to a mammal, especially a human in need of such treatment, prevention, elimination, alleviation or amelioration of various thrombolytic or coagulophatic diseases or disorders as mentioned above. Such mammals also include animals, both domestic animals, e.g. household pets, and non-domestic animals such as wildlife.

Usually, dosage forms suitable for oral, nasal, pulmonal or transdermal administration comprise from about 0.001 mg to about 100 mg, preferably from about 0.01 mg to about 50 mg of the compounds of formula I admixed with a pharmaceutically acceptable carrier or diluent.

The compounds may be administered concurrently, simultaneously, or together with a pharmaceutically acceptable carrier or diluent, whether by oral, rectal, or parenteral (including subcutaneous) route. The compounds are often, and preferably, in the form of an alkali metal or earth alkali metal salt thereof.

Suitable dosage ranges varies as indicated above depending upon the exact mode of administration, form in which administered, the indication towards which the administration is directed, the subject involved and the body weight of the subject involved, and the preference and experience of the physician or veterinarian in charge.

The compounds of the present invention have interesting pharmacological properties. For example, the compounds of this invention can be used to modulate and normalise an impaired haemostatic balance in mammals caused by deficiency or malfunction of blood clotting factors or their inhibitors. The FVIIa and in particular the TF/FVIIa activity plays an important role in the control of the coagulation cascade, and modulators of this key regulatory activity such as the present invention can be used in the treatment of or prophylaxis of thrombotic or coagulopathic related diseases or disorders including vascular diseases and inflammatory responses. The pharmaceutical composition of the invention may thus be useful for modulating and normalising an impaired haemostatic balance in a mammal. In particular, the pharmaceutical composition may be useful for the treatment of or prophylaxis of thrombotic or coagulopathic related diseases or disorders including vascular diseases and inflammatory responses.

“Modulating and normalising an impaired haemostatic balance” means achieving an effect on the coagulation system measurable in vitro assays and/or animal models which diminishes the risk for thrombosis or bleedings.

More particularly, the pharmaceutical composition may be useful as an inhibitor of blood coagulation in a mammal, as an inhibitor of clotting activity in a mammal, as an inhibitor of deposition of fibrin in a mammal, as an inhibitor of platelet deposition in a mammal, in the treatment of mammals suffering from deep venous thrombosis, arterial thrombosis, post surgical thrombosis, coronary artery bypass graft (CABG), percutaneous transdermal coronary angioplastry (PTCA), stroke, tumour metastasis, inflammation, septic chock, hypotension, ARDS, pulmonary embolism, disseminated intravascular coagulation (DIC), vascular restenosis, platelet deposition, myocardial infarction, angiogenesis, or the prophylactic treatment of mammals with atherosclerotic vessels at risk for thrombosis. The compositions of the invention may also be used as an adjunct in thrombolytic therapy.

Furthermore the invention relates to a method for inhibiting the TF initiation activity in a mammal which method comprises administering an effective amount of at least one compound of the present invention, in combination with a pharmaceutical acceptable diluent and/or carrier to the mammal in need of such a treatment.

Assays

Inhibition of FVIIa/Phospholipids-Embedded TF-Catalyzed Activation of FX by TF Antagonists FXa Generation Assay (Assay 1):

In the following example all concentrations are final. Lipidated TF (10 pM), FVIIa (100 pM) and TF antagonist or FFR-rFVIIa (0-50 nM) in HBS/BSA (50 mM hepes, pH 7.4, 150 mM NaCl, 5 mM CaCl₂, 1 mg/ml BSA) are incubated 60 min at room temperature before FX (50 nM) is added. The reaction is stopped after another 10 min by addition of ½ volume stopping buffer (50 mM Hepes, pH 7.4, 100 mM NaCl, 20 mM EDTA). The amount of FXa generated is determined by adding substrate S2765 (0.6 mM, Chromogenix, and measuring absorbance at 405 nm continuously for 10 min. IC₅₀ values for TF antagonist inhibition of FVIIa/lipidated TF-mediated activation of FX may be calculated. The IC50 value for FFR-rFVIIa is 51+/−26 pM in this assay.

Inhibition of FVIIa/Cell Surface TF-Catalyzed Activation of FX by TF Antagonists (Assay 2):

In the following example all concentrations are final. Monolayers of human lung fibroblasts WI-38 (ATTC No. CCL-75) or human bladder carcinoma cell line J82 (ATTC No. HTB-1) or human keratinocyte cell line CCD 1102KerTr (ATCC no. CRL-2310) constitutively expressing TF are employed as TF source in FVIIa/TF catalyzed activation of FX. Confluent cell monolayers in a 96-well plate are washed one time in buffer A (10 mM Hepes, pH 7.45, 150 mM NaCl, 4 mM KCl, and 11 mM glucose) and one time in buffer B (buffer A supplemented with 1 mg/ml BSA and 5 mM Ca²⁺). FVIIa (1 nM), FX (135 nM) and varying concentrations of TF antagonist or FFR-rFVIIa in buffer B are simultaneously added to the cells. FXa formation is allowed for 15 min at 37° C. 50-μl aliquots are removed from each well and added to 50 μl stopping buffer (Buffer A supplemented with 10 mM EDTA and 1 mg/ml BSA). The amount of FXa generated is determined by transferring 50 μl of the above mixture to a microtiter plate well and adding 25 μl Chromozym X (final concentration 0.6 mM) to the wells. The absorbance at 405 nm is measured continuously and the initial rates of colour development are converted to FXa concentrations using a FXa standard curve. The IC50 value for FFR-rFVIIa is 1.5 nM in this assay.

Inhibition of ¹²⁵I-FVIIa Binding to Cell Surface TF by TF Antagonists (Assay 3):

In the following example all concentrations are final. Binding studies are employed using the human bladder carcinoma cell line J82 (ATTC No. HTB-1) or the human keratinocyte cell line (CCD1102KerTr ATCC No CRL-2310) or NHEK P166 (Clonetics No. CC-2507) all constitutively expressing TF. Confluent monolayers in 24-well tissue culture plates are washed once with buffer A (10 mM Hepes, pH 7.45, 150 mM NaCl, 4 mM KCl, and 11 mM glucose) supplemented with 5 mM EDTA and then once with buffer A and once with buffer B (buffer A supplemented with 1 mg/ml BSA and 5 mM Ca²⁺). The monolayers are preincubated 2 min with 100 μl cold buffer B. Varying concentrations of Mabs (or FFR-FVIIa) and radiolabelled FVIIa (0.5 nM ¹²⁵I-FVIIa) are simultaneously added to the cells (final volume 200 μl). The plates are incubated for 2 hours at 4° C. At the end of the incubation, the unbound material is removed, the cells are washed 4 times with ice-cold buffer B and lysed with 300 μl lysis buffer (200 mM NaOH, 1% SDS and 10 mM EDTA). Radioactivity is measured in a gamma counter (Cobra, Packard Instruments). The binding data are analyzed and curve fitted using GraFit4 (Erithacus Software, Ltd., (U.K.). The IC50 value for FFR-rFVIIa is 4 nM in this assay.

Biosensor Assay (Assay 4):

TF antagonists are tested on the Biacore instrument by passing a standard solution of the TF antagonist over a chip with immobilized TF. This is followed by different concentrations of sTF in 10 mM hepes pH 7.4 containing 150 mM NaCl, 10 mM CaCl₂ and 0.0003% polysorbate 20. Kd's are calculated from the sensorgrams using the integrated Biacore evaluation software.

The present invention is further illustrated by the following examples.

The present invention is not to be limited in scope by the specific embodiments disclosed in the examples which are intended as illustrations of a number of aspects of the invention and any embodiments which are functionally equivalent are within the scope of this invention. Those skilled in the art will know, or be able to ascertain using no more than routine experimentation, many equivalents to the specific embodiments of the invention described herein. These and all other equivalents are intended to be encompassed by the following claims.

### EXAMPLES

### Example 1

Generic Construct of Radionuclide Linker Moiety Conjugates C-(LM) for Delivery of TF Antagonist Containing Radionuclides:

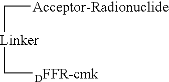

In the schematic illustration, the radionuclide of choice is conjugated to the FVIIa inhibitor D-Phe-Phe-Arg chloromethyl ketone (_{D}FFR-cmk). This may be expanded to all other FVIIa inhibitors. The “linker” refers to other parts of the LM, which separates the compound containing a radionuclide from the FVIIa polypeptide. It is to be understood that the radionuclide linker moiety conjugates C-(LM) comprising the FVIIa inhibitor is reacted with FVIIa polypeptide to get the TF antagonist of the invention. The radionuclide is delivered to or into the TF presenting cell. The acceptor could be any group to which can be coupled a radionuclide using conventional chemistry, e.g. Bolton-Hunter.

### Example 6

Specific Construct of Radionuclide Linker Moiety Conjugates C-(LM) for Delivery of TF Antagonist Containing Radionuclides:

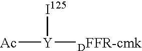

In the schematic illustration, the radionuclide I¹²⁵ is conjugated to the FVIIa inhibitor D-Phe-Phe-Arg chloromethyl ketone (_{D}FFR-cmk). This may be expanded to all other FVIIa inhibitors. It is to be understood that the radionuclide linker moiety conjugates C-(LM) comprising the FVIIa inhibitor is reacted with FVIIa polypeptide to get the TF antagonist of the invention. The radionuclide is delivered to or into the TF presenting cell. The acceptor could be any group to which can be coupled a radionuclide using conventional chemistry, e.g. Bolton-Hunter.

### Example 7

Specific Construct of Radionuclide Linker Moiety Conjugates C-(LM) for Use of TF Antagonist Containing Radionuclides for Diagnostic Imaging of Target Cells:

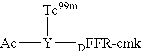

In the schematic illustration, the radionuclide Tc^{m99} is conjugated to the FVIIa inhibitor D-Phe-Phe-Arg chloromethyl ketone (_{D}FFR-cmk). This may be expanded to all other FVIIa inhibitors. It is to be understood that the radionuclide linker moiety conjugates C-(LM) comprising the FVIIa inhibitor is reacted with FVIIa polypeptide to get the TF antagonist of the invention. The radionuclide is delivered to or into the TF presenting cell. The acceptor could be any group to which can be coupled a radionuclide using conventional chemistry, e.g. Bolton-Hunter.

### Example 8

Labeling of rFVIIa with I-123.

200 μg protein in 1000 μl Glycylglycin buffer was labelled with 370 MBq 123I. To obtain high labeling efficiency in the I-123 iodination, the radioactive iodide was diluted with non-radioactive iodide in the form of KI to a total iodide content of 3 nmol. The labeling was achieved using hydrogen peroxide (H₂O₂, 10 μl, 1 mM) and lactoperoxidase (40 μl, 0.1 μg/μl) as oxidizing system.

After iodination, the [123I]rFVIIa formed was separated from unreacted iodine by desalting through a small size-exclusion column (NAP 10). The column was eluted with 1.5 ml vehicle with 0.5% RSA (vehikel: 10 mM Glycylglycin, 150 mM NaCl, 10 mM CaCl2, pH 7.5). The product was further adjusted with vehikel and rFVIIa to the specifications needed. (75 MBq/ml, 1.7 mg/ml rFVIIa, 0.5% RSA, 10 mM Glygylglycin, 150 mM NaCl, 10 mM CaCl2, pH 7.5).

Finally, radiochemical purity was determined on Reverse Phase chromatography on a VYDAC C4 column with in-line monitoring of the radio-signal. The radiochemical purity was >95% with major impurities being protein related.

### Example 9

Labeling of rFVIIa with Other Isotopes of Iodine:

The above procedure is also applicable in the labeling with other isotopes of iodine such as 125I, 124I, 130I, 132I, 135I and could be envisaged in the labeling with other halogen isotopes (bromine, astate).

### Example 10

Labeling of rFVIIa with Other Oxidizing Agents

In addition other oxidizing systems such as Chloramin-T, Iodogen, Iodobeads, iodate, sodium nitrite etc. can be used in lieu of H₂O₂/lactoperoxidase.

### Example 11

Labeling of rFVIIa with 64Cu, 90Y or 111In.

The protein can be derivatized with DTPA, TETA, DOTA or another suitable coordination group, usually in the form of their respective anhydrides. The proteins are separated from unreacted coordination groups by size exclusion chromatography. The resulting derivatized protein is formulated in a suitable buffer and mixed with the radionuclide solution and is left to incubate for 1-2 hours. Depending on labeling efficiency (ie. how much radionuclide is “captured” by the derivatized protein), the resulting solution is either ready for use or must be purified. Separation from unreacted radionuclide can be accomplished using size exclusion chromatography, dialysis, affinity chromatography, anion exchange chromatography etc.

### Example 12

Preparation of [^{99m}Tc(H₂O)₃(CO)₃]⁺.

^{99m}TcO₄⁻ (1 mL, 1 GBq/mL) was added to a vial containing the Isolink™ carbonyl labelling reagent. Isolink™ is composed of the following lyophilised salts: sodium tartrate (8.5 mg), sodium tetraborate (2.85 mg), sodium carbonate (7.15 mg) and sodium boranocarbonate (4.5 mg). The reaction mixture was tightly sealed and stirred (100° C., 25-30 minutes). After reaching room temperature the solution was acidified (pH 4.5-4.75) with HCl (˜2 ml, 0.1 N). The crude product (>85% of [^{99m}Tc(H₂O)₃(CO)₃]⁺ according to HPLC) was employed in the next step without purification. HPLC: Merck Hitachi HPLC and Moelsgaard radioactivity detector, gradient 0-5 min 100% A, 5-6 min 0-25% B, 6-9 min 25-34% B, 9-20 min 34-100% B, Solvent A: TEAP 0.5 M, solvent B: MeOH, Column: Luna C18 250 mm×4.6 mm, 5 μm, Flow 1 mL/min.

Preparation of [^{99m}Tc(CO)₃]rFVIIa.

[^{99m}Tc(H₂O)₃(CO)₃]⁺ (0.5 mL, 0,3 GBq/mL) was added to rFVIIa (1.4 mg/mL, 0.6 mL). The resulting mixture was diluted with saline (0.9% w/w NaCl, 3.1 mL) and the reaction mixture was gently stirred (37° C., 90 minutes). The crude product was desalted using a NAP-10 solid phase extraction column (Glycylglycin 0,1 M NaCl 1.0 M, CaCl 0.1 M, HAS 0.5 w/w %, 1 mL). After desalting the radiochemical purity of [^{99m}Tc(CO)₃]rFVIIa was >90% according to HPLC. HPLC: Merck Hitachi HPLC and Berthold radioactivity detector, gradient 0-30 min 0-100% B, Solvent A: TFA 0.1%, MeCN 10%. B: TFA 0.1% MeCN 90%, Column: Vydac C4 250 mm×4.6 mm, 5 μm, Flow 1 mL/min.

1. A compound having the formula A-(LM)-C, wherein A is a TF antagonist or TF agonist; LM is an optional linker moiety; C is a compound comprising a radionuclide.

2. The compound according to claim 1, wherein A is a TF agonist.

3. The compound according to claim 2, wherein said TF agonist is native human FVIIa or a variant thereof.

4. The compound according to claim 1, wherein A is a TF antagonist.

5. The compound according to claim 4, wherein the TF antagonist is an inactive FVIIa polypeptide.

6. The compound according to claim 5, wherein the inactive FVIIa polypeptide is native human FVIIa or a fragment thereof catalytically inactivated in the active site.

7. The compound according to claim 6, wherein the inactive FVIIa polypeptide is native human FVIIa catalytically inactivated in the active site.

8. The compound according to claim 5, wherein C or (LM)-C is conjugated to the active site of the FVIIa polypeptide.

9. The compound according to claim 5, wherein the FVIIa polypeptide is catalytically inactivated in the active site with a chloromethyl ketone inhibitor independently selected from the group consisting of Phe-Phe-Arg chloromethyl ketone, Phe-Phe-Arg chloromethylketone, D-Phe-Phe-Arg chloromethyl ketone, D-Phe-Phe-Arg chloromethylketone Phe-Pro-Arg chloromethylketone, D-Phe-Pro-Arg chloromethylketone, Phe-Pro-Arg chloromethylketone, D-Phe-Pro-Arg chloromethylketone, L-Glu-Gly-Arg chloromethylketone and D-Glu-Gly-Arg chloromethylketone, Dansyl-Phe-Phe-Arg chloromethyl ketone, Dansyl-Phe-Phe-Arg chloromethylketone, Dansyl-D-Phe-Phe-Arg chloromethyl ketone, Dansyl-D-Phe-Phe-Arg chloromethylketone, Dansyl-Phe-Pro-Arg chloromethylketone, Dansyl-D-Phe-Pro-Arg chloromethylketone, Dansyl-Phe-Pro-Arg chloromethylketone, Dansyl-D-Phe-Pro-Arg chloromethylketone, Dansyl-L-Glu-Gly-Arg chloromethylketone and Dansyl-D-Glu-Gly-Arg chloromethylketone.

10. The compound according to claim 5, wherein LM comprises a chloromethyl ketone inhibitor independently selected from the group consisting of Phe-Phe-Arg chloromethyl ketone, Phe-Phe-Arg chloromethylketone, D-Phe-Phe-Arg chloromethyl ketone, D-Phe-Phe-Arg chloromethylketone Phe-Pro-Arg chloromethylketone, D-Phe-Pro-Arg chloromethylketone, Phe-Pro-Arg chloromethylketone, D-Phe-Pro-Arg chloromethylketone, L-Glu-Gly-Arg chloromethylketone and D-Glu-Gly-Arg chloromethylketone, Dansyl-Phe-Phe-Arg chloromethyl ketone, Dansyl-Phe-Phe-Arg chloromethylketone, Dansyl-D-Phe-Phe-Arg chloromethyl ketone, Dansyl-D-Phe-Phe-Arg chloromethylketone, Dansyl-Phe-Pro-Arg chloromethylketone, Dansyl-D-Phe-Pro-Arg chloromethylketone, Dansyl-Phe-Pro-Arg chloromethylketone, Dansyl-D-Phe-Pro-Arg chloromethylketone, Dansyl-L-Glu-Gly-Arg chloromethylketone and Dansyl-D-Glu-Gly-Arg chloromethylketone, wherein the inactive FVIIa polypeptide is catalytically inactivated in the active site with said chloromethyl ketone inhibitor.

11. The compound according to claim 4, wherein the TF antagonist is an antibody against TF.

12. The compound according to claim 11, wherein the antibody is a human monoclonal antibody against human TF.

13. The compound according to claim 1, wherein C is a compound containing the radionuclide selected from the list consisting of Tc-99m, Iodine-123, Iodine-131, Indium-111, and Fluorine-18.

14. The compound according to claim 1, wherein C comprises a protein or peptide.

15. The compound according to claim 1, wherein C or (LM)-C is conjugated at the glycosylation side chains of A.

16. The compound according to claim 1, wherein C or (LM)-C is conjugated to a free sulfhydryl group present on A.

17. The compound according to claim 1, wherein the compound comprises more than one binding site for TF.

18. The compound according to claim 1, wherein LM comprises an amino acid sequence.

19. The compound according to claim 18, wherein LM comprises an amino acid sequence of Gly-Gly.

20. The compound according to claim 1, wherein LM comprises a molecule selected from the group consisting of straight or branched C₁₋₅₀-alkyl, straight or branched C₂₋₅₀-alkenyl, straight or branched C₂₋₅₀-alkynyl, a 1 to 50-membered straight or branched chain comprising carbon and at least one N, O or S atom in the chain, C₃₋₈cycloalkyl, a 3 to 8-membered cyclic ring comprising carbon and at least one N, O or S atom in the ring, aryl, heteroaryl, amino acid, the structures optionally substituted with one or more of the following groups: H, hydroxy, phenyl, phenoxy, benzyl, thienyl, oxo, amino, C₁₋₄-alkyl, —CONH₂, —CSNH₂, C₁₋₄ monoalkylamino, C₁₋₄ dialkylamino, acylamino, sulfonyl, carboxy, carboxamido, halogeno, C₁₋₆ alkoxy, C₁₋₆alkylthio, trifluoroalkoxy, alkoxycarbonyl, and haloalkyl.

21. The compound according to claim 1, wherein LM comprises a chemical bond, which can broken by chemical reduction.

22. The compound according to claim 21, wherein LM comprises a disulphide bond.

23. The compound according to claim 22, wherein the disulphide bond is between two cysteines.

24. The compound according to claim 1, wherein LM comprises a cleavage site for protease hydrolysis.

25. The compound according to claim 24, wherein the protease is selected from the group consisting of cathepsin B, cathepsin D, cathepsin E, cathepsin G, cathepsin H, cathepsin L, cathepsin N, cathepsin S, cathepsin T, cathepsin K, and legumain.

26. The compound according to claim 25, wherein the protease is cathepsin B.

27. The compound according to claim 26, wherein LM comprises the amino acid sequence Phe-Arg.

28. A pharmaceutical composition comprising an amount of the compound having the formula A-(LM)-C, wherein A is a TF antagonist or TF agonist; LM is an optional linker moiety; C is a compound comprising a radionuclide; and a pharmaceutically acceptable carrier or diluent.

29. A method of inhibiting TF function comprising contacting TF-bearing cells with an effective amount of a compound having the formula A-(LM)-C, wherein A is a TF antagonist or TF agonist; LM is an optional linker moiety; C is a compound comprising a radionuclide.

30. The method of claim 29, wherein the method is applied to treat a disease or disorder associated with pathophysiological TF function in a subject.

31. The method of claim 30, wherein the disease or disorder is selected from bleedings, deep venous thrombosis, arterial thrombosis, post surgical thrombosis, coronary artery bypass graft (CABG), and percutaneous transdermal coronary angioplastry (PTCA).

32. The method of claim 30, wherein the disease or disorder is stroke.

33. The method of claim 30, wherein the disease or disorder is cancer, tumor growth, or tumour metastasis.

34. The method of claim 30, wherein the disease or disorder is rheumatoid arthritis.

35. The method of claim 30, wherein the disease or disorder is a condition involving angiogenesis, ischemia/reperfusion, thrombolysis, or arteriosclerosis and restenosis following angioplastry.

36. The method of claim 30, wherein the disease or disorder is inflammation.

37. The method of claim 30, wherein the disease or disorder is selected from septic chock, septicemia, hypotension, adult respiratory distress syndrome (ARDS), disseminated intravascular coagulopathy (DIC), pulmonary embolism, platelet deposition, myocardial infarction, and prophylactic treatment of mammals with atherosclerotic vessels at risk for thrombosis.

38. The method of claim 29, wherein the method is used to diagnose a subject for a disease or condition associated with pathophysiological TF function.

39. The method of claim 29, wherein the method is used to prevent the development of a disease or condition associated with pathophysiological TF function in a subject.


SEQUENCE LISTING (1 sequence)

SEQ ID NO: 1 - protein, 406 aa, homo Sapiens
  MISC_FEATURE (6)..(7): Xaa=GLA
ANAFLXXLRPGSLXRXCKXXQCSFXXARXIFKDAXRTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP